(19) United States
(12) Patent Application Publication
(10) Pub. No.: US 2013/0280715 A1
(43) Pub. Date: Oct. 24, 2013

(54) Methods, Systems and Compositions for Nucleic Acid Analysis Using Back-Scattering Interferometry

(71) Applicants: Darryl J. Bornhop, Nashville, TN (US); Robert A. Flowers, Nazareth, PA (US)

(72) Inventors: Darryl J. Bornhop, Nashville, TN (US); Robert A. Flowers, Nazareth, PA (US)

(73) Assignee: Vanderbilt University, Nashville, TN (US)

(21) Appl. No.: 13/879,523

(22) PCT Filed: Oct. 13, 2011
(86) PCT No.: PCT/US11/56171

§ 371 Date: Jun. 28, 2013

Related U.S. Application Data
(60) Provisional application No. 61/392,890, filed on Oct. 13, 2010.

(51) Int. Cl. C12Q 1/68

(52) U.S. Cl. CPC C12Q 1/6827; G06F 19/20
USPC 435/6.11; 435/287.2; 702/20

(57) ABSTRACT

Disclosed are methods, systems, and apparatuses for the measurement of hybridization of nucleic acid polymers or binding other biological molecular species such as proteins, enzymes, receptors, and antibodies to binding partners, by backscattering interferometry (BSI).

### RELATED APPLICATION

This application claims the priority of U.S. Provisional Patent Application No. 61/392,890, filed Oct. 13, 2010, which is herein incorporated in its entirety.

### STATEMENT REGARDING FEDERALLY SPONSORED RESEARCH

This invention was made with government support under the National Science Foundation CHE 0848788. The government has certain rights in the invention.

### BACKGROUND

The invention of polymerase chain reaction (PCR) by Kary Mullis in 1984 is considered a revolution in science that has allowed for detecting and quantifying DNA sequences and expression profiles of genes. Many applications of the original PCR procedures have been made, and the field of genomics has been expanded. For example, applications in medicine have yielded information about diseases, pathogens and genetic linkages, including biochemical and biophysical processes underlying the phenotypic expression of cell regulation. In order to fully understand the regulation of metabolism and to alter it successfully, more information of gene expression, recognition of DNA by proteins, transcription factors, drugs and other small molecules is required.

Gene expression profiles have been widely used to address the relationship between ecologically influenced or disease phenotypes and cellular expression patterns. PCR-based detection technologies utilizing specific primers are providing useful as research tool providing enhanced information on biology of all types of organisms and their interactions with each other and other species. For example, PCR is used quite extensively used to detect the presence of numerous disease-causing organisms. PCR can also be used to look for the causes of aseptic meningitis and encephalitis whether it is virus, bacteria, or parasites. PCR-based detection is used in screening for cancer, such as prostate and colorectal cancer Traditionally PCR based methods have relied upon gels with dye-labeled DNA to probe reaction completion and progress, fluorescent labels or double-stranded DNA intercalation dyes.

Some efforts have focused on methods of detection of DNA with electrochemistry showing some promise but still requiring immobilization and a ‘label’ (Yeung, S., et al. 2008). An approach to “reagentless” PCR has also been published which claims the ability to measure conformational changes and thus sequence-specific detection (Fan, C., et al. 2003). Sensitivity is limited when gels are employed thus the procedures usually require numerous cycles to generate enough copies of DNA to be detected. Real-time PCR methods are more widely used, but still require the use of a fluorophore label. Recently reported electrochemical based methods appear to be able to detect DNA in a fewer number of cycles, but require additional chemistry that involves labeling the probe DNA to make it electro-active and/or some type of surface immobilization approach.

Miniaturization and on-chip PCR embodiments are also used which use labeling the probe for detection and/or simply for quantification of a single predetermined species. In many cases on-chip methods require relatively high number of cycles of PCR due to sensitivity limitations of the detection system, or demand an expensive detection approach. Thus, there remains a need in the art for improved methods of detecting nucleic acid hybridization in free solution.

### BRIEF SUMMARY

The present invention comprises methods of using backscattering interferometry (BSI) to detect the complementary binding or hybridization of nucleic acid polymers, for example, to detect mismatching of nucleic acid bases. One or more nucleic acid polymers may be the products of PCR amplification, or may be any nucleic acid from a sample. Methods comprise using one or more nucleic acid probes of a known sequence (first nucleic acid polymer) to detect at least a second nucleic acid polymer, wherein the first nucleic acid polymer may be the complement of the second polymer, or may differ by one, two or more bases from the second polymer, or may have a sequence that differs by mismatch bases, deleted bases, added bases, rearranged bases, modified bases, or has inserted or reversed sequences.

The present invention comprises increased speed in detection of nucleic acids in sample when compared to PCR amplification methods, and if PCR is used to generate nucleic acids in a sample, the entire PCR may comprise fewer cycles. Methods of the present invention may not use fluorescent or electrochemical labels on nucleic acid polymers, and the methods may comprise small sample volumes, and high sensitivity. Methods of the present invention comprise detection of DNA, RNA and nucleic acid polymers having modified bases. The nucleic acid polymers may be free in solution or may be bound to a substrate.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a graph showing free-solution BSI average plots of oligonucleotide hybridization at equilibrium with a constant probe strand (Ps) oligomer concentration of 150 nM and a varied concentration (0-300 nM) of unlabeled, Cy3-labeled or FITC-labeled, terminal or internal mismatched, or nonsense strand.

FIG. 2 is a graph showing surface-immobilized BSI 5 trial average plots of hybridized unlabeled, Cy3-labeled, FITC-labeled, terminal mismatch, internal mismatch, and nonsense oligonucleotides ranging from 0-250 nM.

FIG. 3 is a bar graph showing a direct comparison of surface immobilization and free-solution oligonucleotide hybridization was carried out using back-scattering interferometry (BSI). Comparison of both formats shows that surface immobilization significantly perturbs hybridization altering it by as much as 50%. BSI can be used to distinguish between a perfect complement and a 2 base pair mismatch with an internal mismatch significantly destabilizing the duplex.

FIG. 4 is a schematic of an exemplary inferometry device, a BSI device.

FIG. 5 is a bar graph showing the change in absolute signal for mouse actin DNA hybridization reactions using BSI. ssDNA corresponds to a single strand DNA immobilized on the surface and PBS buffer present in the channel; cDNA corresponds to complete hybridization reaction when ssDNA and its complimentary cDNA are on the surface and PBS buffer is in the channel.

FIG. 6 is a bar graph showing the change in the signal produced by repetitive hybridization and denaturation of mouse actin DNA molecules immobilized on the surface. Variation in the signal between runs can be attributed to incomplete cDNA removal.

FIG. 7 is a graph of BSI data from a first PCR experiment.

FIG. 8 is a graph of BSI data from a second PCR experiment samples. Channel emptied between sample injections.

FIG. 9 is a graph of phase wrap and correcting for it in a DMSO calibration curve.

FIG. 10 is a graph of phase corrected BSI data from PCR samples. Note the scale on the Y axis which corresponds to an extremely large change in RI.

FIG. 11 is a graph of BSI data from a third PCR experiment.

FIG. 12 is a graph of raw BSI data from a third PCR experiment.

FIG. 13 is a graph of phase corrected BSI data from a third PCR experiment.

FIG. 14 is a graph of phase corrected BSI data from PCR samples with controls out to 30 cycles.

FIG. 15 is a graph showing free-solution BSI unlabeled oligonucleotide duplex calibration experiments.

FIG. 16 is a graph showing free-solution BSI unlabeled oligonucleotide duplex calibration experiments.

FIG. 17 is a graph of binding curves of DNA by BSI detection.

FIG. 18 is a graph of BSI detection of short strands of RNA.

FIG. 19 is a graph of BSI detection of full-length respiratory syncytial virus (RSV) N-gene.

FIG. 20 is a graph of BSI detection of 1200 mer RNA using multiple probes.

FIG. 21 is a schematic of BSI detection using multiple immobilized probes.

FIG. 22 is a graph of BSI detection by free solution and immobilized probes.

FIG. 23A-D shows the single channel sample-reference BSI compensates for large RI changes. A shows the regions of the elongated fringe pattern interrogated. B shows RI compensation for temperature changes. C shows RI compensation for changes in concentration. D shows an illustration of the detection regions to be illuminated by an expanded beam in the single-channel multiplex design.

FIGS. 24 A and B shows images of the fringe patterns from a single-channel multiplex experiment. A shows a screen shot of fringe image output and the regions of the fringes that correspond to the different samples. B shows the corresponding line profile of the fringes to be used in the FFT analysis.

FIG. 25 is a graph of a free-solution calibration curve using the single-channel multiplex design, where one sensing region contained water and was used as an internal reference while the second region contained increasing concentrations of glycerol.

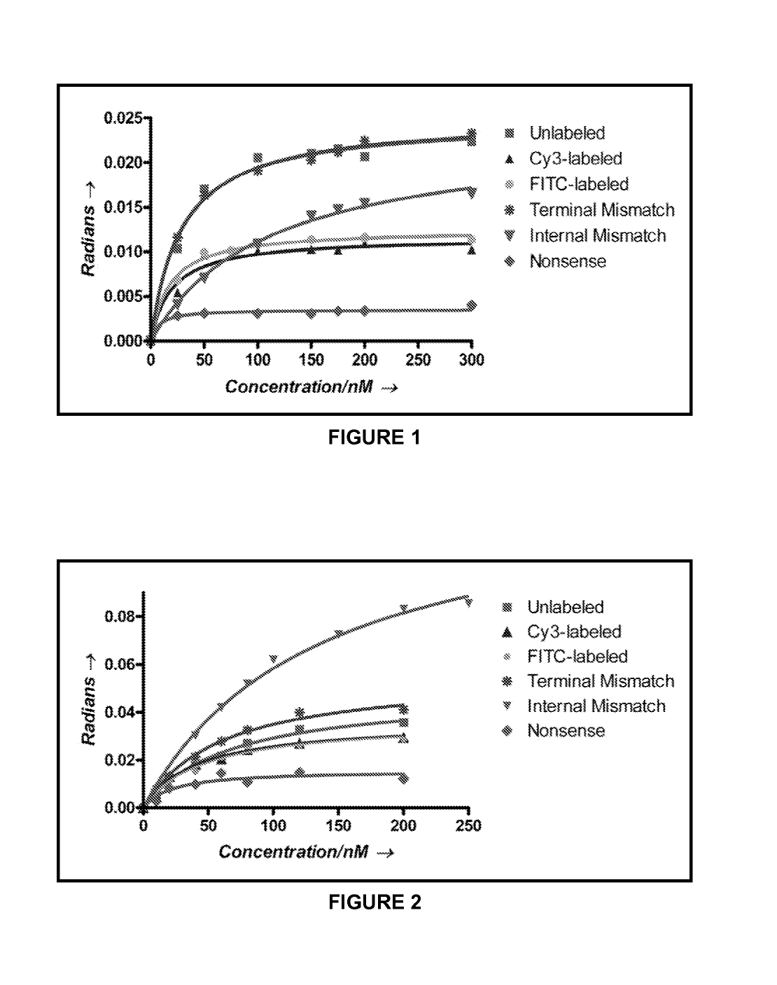

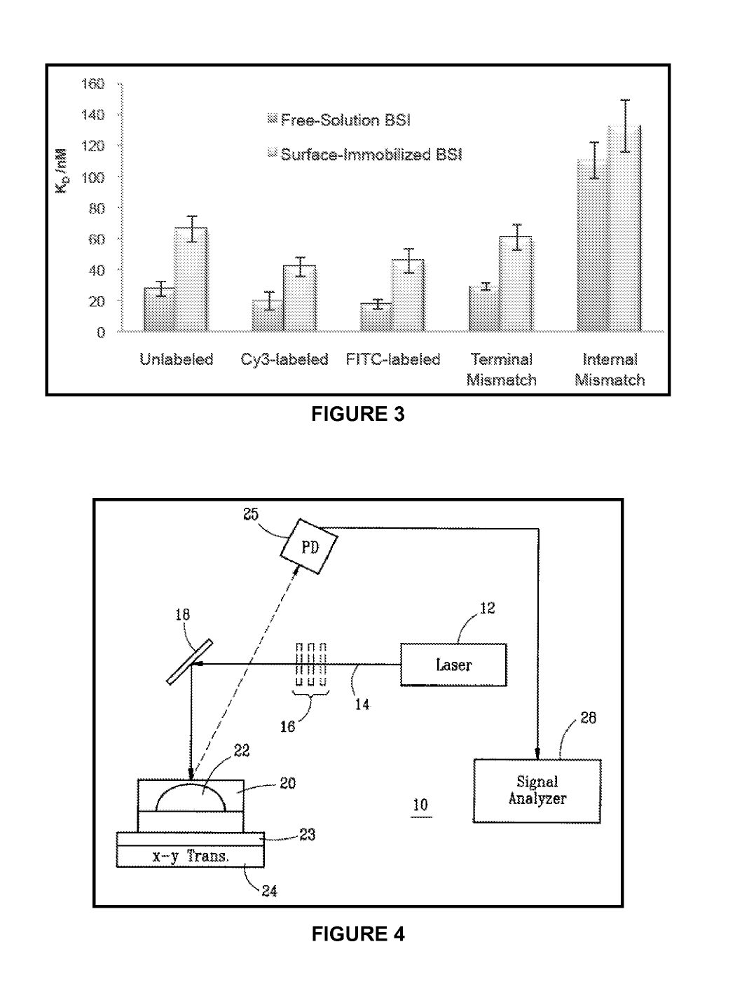

### DETAILED DESCRIPTION

The present invention comprises methods, systems and compositions for detection of nucleic acids, and nucleic acid interactions, using backscattering interferometry. Quantification of binding interactions is useful for understanding cellular processes in biological systems and is important in diagnostics and drug development. One of the major problems in the study of biomolecular interactions is the need to introduce a fluorescent label, or surface attachment, in order to study the system. These alterations perturb the system, but the extent of perturbation, while recognized, is often not clear because the system cannot be studied in the absence of a modification using the same experimental apparatus. This is especially important in the study of DNA hybridization where surface immobilization and fluorescent tags are routinely employed.

Back-scattering interferometry (BSI) can be used to effectively monitor molecular binding interactions both in free-solution and in a surface-immobilized format. Examples herein show that: (1) Surface immobilization may perturb hybridization by as much as 50%, (2) BSI can be used to distinguish between a perfect complement and base pair mismatch, for example one or more base pair mismatches, with an internal mismatch detectable in that the mismatch destabilizes the duplex, and (3) a terminal fluorophore has some impact on DNA hybridization. The present invention is broadly applicable to a range of biomolecular interactions, particularly nucleic acid binding reactions.

The reversible nature of DNA hybridization is critical to cellular replication and protein transcription. Hybridization assays may be carried out using fluorometry, surface plasmon resonance (SPR), or interferometry. Although these techniques have vastly improved the study of nucleotide hybridization, generally they require either fluorescent labeling or surface immobilization for signal generation. Studies designed to determine the effects of fluorophore attachment on DNA duplex hybridization and stability have shown that the strand location and structure of the fluorophore are important factors that determine their impact. To date, it has been difficult to directly compare hybridization efficacies for surface-immobilizing versus free-solution techniques since detection platforms are specifically designed for a single sensing format. Previous work performed to address this problem has employed isotopic assays, duplex melting, and kinetic hybridization studies to analyze the impact of immobilization on DNA hybridization. These studies have shown that oligonucleotide immobilization can perturb hybridization while the density of the immobilized probe was a critical feature in duplex stability and reproducibility of results. The most common method to interrogate duplex stability for surface-immobilized experiments is to carry out duplex melting for the surface-immobilized DNA and compare to bulk solution. However this method is not a direct comparison nor does it always reflect the affinity at lower temperatures in which the hybridization experiment normally takes place. Methods of the present invention comprising BSI can make such a comparison.

BSI monitors a change in refractive index to determine the binding affinity of molecular interactions. In general, this technique may comprise a He—Ne laser focused perpendicularly onto a microfluidic channel in a glass chip to generate a back-scattered interference fringe pattern. The introduction of two binding pairs, for example, two nucleic acid molecules, into the channel creates a change in refractive index, causing a spatial shift in the fringe pattern. The magnitude of this shift depends on which fringes are interrogated, the concentration of the binding pairs, conformational changes initiated upon binding, changes in water of hydration, and binding affinity. Unlike many other surface-immobilizing interferometric techniques, BSI can also measure binding in free-solution as long as the channel surface remains constant. This makes BSI the only technique that can determine binding affinities in a label-free manner without relying on thermal changes or surface immobilization. Additionally, BSI has significantly lower detection limits than other free-solution techniques, allowing experiments to be carried out with physiologically relevant concentration ranges. Use of BSI is generally described in U.S. Pat. Nos. 5,325,170; 6,381,025; 6,809,828; and 7,130,060, each of which is incorporated in its entirety.

As disclosed herein, methods comprising backscattering interferometry (BSI) can circumvent the limitations involved in detection of nucleic acid hybridization, allowing the realization of the full advantages of microfluidics. BSI methods herein provide a label-free, free-solution or direct on-chip, highly sensitive detection technique. BSI methods disclosed herein detect nucleic acid hybridization, the products of PCR amplification with fewer cycles of PCR needed, and can provide detection without the use of fluorescent or electrochemical labels. Detection of nucleic acid hybridization by BSI methods and devices can be used for gene or genome detection or identification and can detect the location of sequence identity or mismatch sequences, a mutation in a target sequence, and without the need for sequencing the target sequence. BSI nucleic acid hybridization detection has the ability to measure molecular interactions directly in free solution or on-chip in, for example, nanoliter volumes and at zeptomole sensitivity.

Back scattering interferometry (BSI) methods and devices allow for the study of molecular interactions on a smaller scale than other commonly-used techniques, generally needing only a microliter of sample per trial. In addition, BSI methods and devices can be used to study the interactions of large molecules such as DNA without the interference of a fluorophore tag or a surface-immobilized sample. Instead, DNA binding interactions can be performed in free solution, without the interference of other components such as fluorophores.

In general, BSI methods and devices measure the change in refractive index of a solution as two molecules bind. A back-scattered interference fringe pattern is generated when the beam of a laser is directed through a microfluidic chip containing the sample solution and then reflected back to a CCD detector. The hybridization of nucleic acid molecules (strands) are interrogated by BSI methods and devices, and binding curve data may be analyzed using computer methods, such as those provided by Microsoft Excel and GraphPad Prism 4. A report is generated of the results of the BSI methods and devices. Binding curves and K_{D} values for the strands' interactions are obtained using the BSI device. Once a complementary binding curve was obtained by hybridizing two nucleic acid molecules that are substantially complementary at every base pair, the hybridization of other strands may be performed, and data, including K_{D} values, is generated. For example, single nucleotide polymorphism (SNPs; either a mismatched or deleted base pair) may be examined. New binding curves for each SNP can be compared to that of the complementary strand using the K_{D} values in order to gauge how the SNP affects the hybridization. A lower K_{D} corresponds to tighter binding, thus, a strand that was not a perfect complement to the known nucleic acid strand would be expected to have a higher K_{D} value than the complementary strand, since the SNP would disrupt and weaken the strands' interactions. The hybridization binding data can be obtained for all types of nucleic acids, and can be used to interrogate the sequences of such nucleic acid molecules without having to ascertain the identity of each nucleotide individually by sequencing methods.

### Interferometric Detection

In an aspect, the invention relates to the use of an interferometric detection system for detecting nucleic acids and nucleic acid interactions in a sample, for example, for detection of refractive index (RI) changes in picoliter sized sample volumes. A system includes a source of coherent light, such as a diode or He—Ne laser, a channel of capillary dimensions that is preferably etched or molded in a substrate for reception of a sample to be analyzed, and a photodetector for detecting backscattered light from the sample at a detection zone, and may further comprise computer means for interpreting the photodetector signals or manipulating and/transferring data, sample injection and removal means, or automated components for manipulating samples.

In an aspect, the laser source generates an easy to align simple optical train comprised of collimated laser beam that is incident on the etched channel (or capillary) for generating the backscattered light. The backscattered light comprises interference fringe patterns that result from the reflective and refractive interaction of the incident laser beam with the channel walls and the sample. These fringe patterns include a plurality of light bands whose positions shift as the refractive index of the sample is varied, either through compositional changes or through temperature changes, for example. The photodetector detects the backscattered light and converts it into intensity signals that vary as the positions of the light bands in the fringe patterns shift, and can thus be employed to determine the refractive index (RI), or an RI related characteristic property, of the sample. A signal analyzer, such as a computer or an electrical circuit, is employed for this purpose to analyze the photodetector signals, and determine the characteristic property of the sample.

In an aspect, the channel has a generally semi-circular cross-sectional shape. A unique multi-pass optical configuration is inherently created by the channel characteristics, and is based on the interaction of the unfocused laser beam and the curved surface of the channel, that allows interferometric measurements in small volumes at high sensitivity. Additionally, if a laser diode is employed as the source, not only does this enable use of wavelength modulation for significant improvements in signal-to-noise ratio, but it also enables integration of the entire detector device directly onto a single microchip.

Alternatively, the channel can have a substantially circular or generally rectangular cross-sectional shape. In an aspect, the substrate and channel together comprise a capillary tube. In an aspect, the substrate and channel together comprise a microfluidic device, for example, a silica substrate, or a polymeric substrate [e.g., polydimethylsiloxane (PDMS) or polymethyl methacrylate (PMMA)], and an etched channel formed in the substrate for reception of a fluid sample, the channel having a cross sectional shape. It is also contemplated that the substrate can further comprise a reference channel. For example, the detection system can employ a second channel, which can comprise a capillary or an on-chip channel of semi-circular or rectangular cross-section.

In an aspect, the sample is a liquid, which can be a substantially pure liquid, a solution, or a mixture (e.g., biological fluids, cellular fluids). In an aspect, the fluid can further comprise one or more nucleic acid polymers.

Free-solution determination methods for molecular interactions are contemplated by the present invention.

Techniques that observe immobilized nucleic acid polymers may limit conformational and translational freedom for nucleic acid polymers whereas free-solution analysis techniques mimic in vivo measurements because nucleic acid polymers enjoy unrestricted freedom in all three dimensions during measurement. Alternatively, methods comprising immobilization of probes may allow for increased signal detection.

In an aspect, the invention relates to a method comprising detecting molecular interaction between nucleic acid polymers in free-solution wherein one or more of the nucleic acid polymers are label-free and detection is performed by back-scattering interferometry (BSI). In an aspect, the nucleic acid polymers are present in a sample in a microfluidic channel in a substrate. In an aspect, back-scattering interferometry comprises directing a coherent light beam onto the substrate such that the light beam is incident on the channel to generate backscattered light through reflective and refractive interaction of the light beam with a substrate channel interface and the sample, the backscattered light comprising interference fringe patterns including a plurality of spaced light bands whose positions shift in response to changes in the refractive index of the fluid sample.

It is contemplated that methods of the present invention can be used to determine one or more of an equilibrium constant, a dissociation constant, a dissociation rate, a dissociation rate constant, an association rate, and/or an association rate constant of the interaction.

Each of one or more nucleic acid polymers can be introduced into the channel in a sample. Two or more nucleic acid polymers can be present in the same or in different samples. Each of the one or more nucleic acid polymers can independently be present in a suitable concentration, for example, a concentration of less than about 1.0×10⁻⁵M, less than about 5.0×10⁻⁶M, less than about 1.0×10⁻⁶M, less than about 5.0×10⁻⁷M, less than about 1.0×10⁻⁷M, less than about 5.0×10⁻⁸M, less than about 1.0×10⁻⁸M, less than about 5.0×10⁻⁹M, or less than about 1.0×10⁻⁹M, less than about 5.0×10⁻¹⁰ M, less than about 1.0×10⁻¹⁰M, less than about 5.0×10⁻¹¹M, less than about 1.0×10⁻¹¹M, less than about 5.0×10⁻¹²M, less than about 1.0×10⁻¹²M, less than about 5.0×10⁻¹³M, less than about 1.0×10⁻¹³M, less than about 1.0×10⁻¹⁴M, less than about 1.0×10⁻¹⁴M, less than about 1.0×10⁻¹⁵M, or less than about 1.0×10⁻¹⁵M.

In an aspect, the interaction can be a biomolecular interaction. For example, two nucleic acid polymers can associate to provide an interaction product. In an aspect, nucleic acid polymers can dissociate to provide two or more interaction products. In further aspects, more than two nucleic acid polymers can be involved in the interaction.

In an aspect, the invention relates to a method for free-solution determination of molecular interactions comprising providing a substrate having a channel formed therein for reception of a fluid sample to be analyzed; introducing a first sample comprising a first non-immobilized nucleic acid polymer to be analyzed into the channel; introducing a second sample comprising a second non-immobilized nucleic acid polymer to be analyzed into the channel; allowing the first nucleic acid polymer to interact, e.g., hybridize with the second nucleic acid polymer to form one or more interaction products; for example, at an hybridization equilibrium, directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; detecting positional shifts in the light bands; and determining the formation of the one or more interaction products of the first nucleic acid polymer with the second nucleic acid polymer from the positional shifts of the light bands in the interference patterns. Pre- and post-processing steps, such as purification of nucleic acid polymers, labeling, PCR, DNA or RNA preparatory steps, such as controlling polymer size, may occur and may be included in the methods disclosed herein.

In an aspect, the invention relates to a method for surface immobilized determination of molecular interactions comprising providing a substrate having a channel formed therein for reception of a fluid sample to be analyzed; immobilizing one or more nucleic acid molecules (a first nucleic acid polymer) to the surface of the channel; introducing a sample comprising a non-immobilized nucleic acid polymer to be analyzed into the channel; allowing the first nucleic acid polymer to interact, e.g., hybridize with the second nucleic acid polymer to form one or more interaction products; for example, at an hybridization equilibrium, directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; detecting positional shifts in the light bands; and determining the formation of the one or more interaction products of the first nucleic acid polymer with the second nucleic acid polymer from the positional shifts of the light bands in the interference patterns. A first nucleic acid polymer may be one or more probe(s) and a non-immobilized nucleic acid polymer may be a target nucleic acid polymer. Pre- and post-processing steps, such as purification, labeling, PCR, DNA or RNA preparatory steps, such as polymer sizing, may occur and may be included in methods disclosed herein. As shown in the examples, and disclosed herein, immobilized and non-immobilized nucleic acid polymer methods are contemplated by the present invention, and the disclosed methods herein may comprise immobilized or non-immobilized nucleic acid polymers.

The methods of the present invention can determine the interaction between one or more nucleic acid polymers by monitoring, measuring, and/or detecting the formation and/or steady state relative abundance of one or more nucleic acid polymer interaction products from the interaction of the one or more nucleic acid polymers. The determination can be performed qualitatively or quantitatively. Interaction rate information can be derived from various measurements of the interaction.

In an aspect, a first sample is combined with a second sample prior to introduction to the BSI device. That is, the nucleic acid polymers are combined (and potentially interacting) prior to performing the disclosed methods. In this aspect, the step of introducing the first nucleic acid polymer and the step of introducing the second nucleic acid polymer are performed simultaneously.

In an aspect, a first sample is combined with a second sample after introduction. That is, the nucleic acid polymers can be combined at a point before the channel, or at a point within the channel, when performing the disclosed methods. In this aspect, the step of introducing the first nucleic acid polymer and the step of introducing the second nucleic acid polymer are performed either simultaneously or sequentially. In an aspect, the detecting step is performed during the interaction of the first nucleic acid polymer with the second nucleic acid polymer.

A method of the present invention comprises end-point measurement, or measurement at equilibrium of an interaction between two nucleic acid polymers. That is, the method can determine the occurrence and/or equilibrium of an interaction between two or more nucleic acid polymers. The present invention may detect the intramolecular hybridization within one nucleic acid polymer, and/or the intermolecular hybridization between two or more nucleic acid molecules. The present invention comprises a method for free-solution determination of molecular interactions comprising providing a substrate having a channel formed therein for reception of a fluid sample to be analyzed; introducing a first sample comprising a first non-immobilized nucleic acid polymer into the channel; establishing a baseline interferometric response by directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the first sample. introducing a second sample comprising a mixture of a first non-immobilized nucleic acid polymer and a second non-immobilized nucleic acid polymer into the channel, which may or may not displace the first sample, or providing a second sample comprising a first nucleic acid and a third sample comprising a second nucleic acid molecule, into the channel; wherein the first nucleic acid polymer interacts with the second nucleic acid polymer to form one or more interaction products; directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the second sample; detecting positional shifts in the light bands relative to the baseline; and determining the formation of the one or more interaction products of the first nucleic acid polymer with the second nucleic acid polymer from the positional shifts of the light bands in the interference patterns. It is to be understood that once a baseline interferometric response is established, that step need not be repeated every time the method is performed with the same nucleic acid molecules. Thus, the method comprises optionally establishing a baseline interferometric response, for example, for the first iteration of the measurement for a particular hybridization reaction. Once the baseline is established, the method can continue with re-establishing the baseline at each further iteration of the method.

A method of the present invention comprises end-point measurement, or measurement at equilibrium of an interaction between two nucleic acid polymers. That is, the method can determine the occurrence and/or equilibrium of an interaction between two or more nucleic acid polymers. The present invention may detect the intramolecular hybridization within one nucleic acid polymer, and/or the intermolecular hybridization between two or more nucleic acid molecules. The present invention comprises a method for determination of molecular interactions comprising providing a substrate having a channel formed therein for reception of a fluid sample to be analyzed wherein at least a first nucleic acid polymer is immobilized in the channel; a) adding a first sample comprising nucleic acid polymers having the same or a known different sequence as the first nucleic acid polymer, and establishing a baseline interferometric response by directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the first sample, and establishing the K_{D} for the hybridization kinetics of the at least two nucleic acid polymers; b) introducing a sample comprising a nucleic acid molecule of interest into the channel, which may or may not displace the first sample, wherein the immobilized first nucleic acid polymers interact with the second nucleic acid polymers to form one or more interaction products; directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the second sample; detecting positional shifts in the light bands relative to the baseline; and determining the formation of the one or more interaction products of the first nucleic acid polymer with the second nucleic acid polymer from the positional shifts of the light bands in the interference patterns, optionally, establishing the K_{D} of the hybridization kinetics. It is to be understood that once a baseline interferometric response is established, that step need not be repeated every time the method is performed with the same nucleic acid molecules. Thus, the method comprises optionally establishing a baseline interferometric response, for example, for the first iteration of the measurement for a particular hybridization reaction. Once the baseline is established, the method can continue with re-establishing the baseline at each further iteration of the method.

From the data of the BSI methods and devices, such as the K_{D} of the hybridization of two or more nucleic acid molecules, a report may be generated. An article of the present invention comprises a report stating the data generated by BSI methods and/or devices of the present invention. A report may state information about the studies performed, the methods employed, the devices used, or other data that can be reported. A method of the present invention comprises performed the hybridization method disclosed herein with a device disclosed herein to yield a report stating the data obtained in such method. As used herein and as is commonly understood by those skilled in the art, a known nucleic acid molecule is one in which the sequence is known or the source of the nucleic acid molecule is known, such as a DNA nucleic acid molecule wherein the sequence identity is known, or a DNA nucleic acid molecule that was isolated from a particular source, such as a cell or microorganism. An unknown nucleic acid molecule is one that may be present in a sample and its presence in the sample may be detected by the methods disclosed herein, or may be a nucleic acid molecule for which the sequence identity has not been determined.

A sample (e.g., a composition comprising a non-immobilized nucleic acid polymer) can be introduced into the channel of the substrate, i.e., a BSI device. The sample can be provided having a known concentration of the nucleic acid polymer.

A baseline interferometric response maybe established for a sample having a known or unknown nucleic acid molecules by directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the first sample.

A sample, which may comprise one or more nucleic acid molecules, e.g., a composition comprising a mixture of a first non-immobilized nucleic acid polymer and a second non-immobilized nucleic acid polymer, wherein the first nucleic acid polymer interacts with the second nucleic acid polymer to form one or more interaction products; or a composition comprising a known or unknown target nucleic acid polymer (which may be referred to herein as a nucleic acid molecule of interest); or a composition comprising a probe, can be introduced into the channel. In various aspects, the sample can be provided as a pre-mixed sample of a first non-immobilized nucleic acid polymer and a second non-immobilized nucleic acid polymer or provided by adding a sample comprising the second non-immobilized nucleic acid polymer to the first sample present in the BSI device. In an aspect, the first sample is a solution of the first nucleic acid polymer, which is displaced in the channel by the introduction of the second sample, which is a solution of both the first nucleic acid polymer and the second nucleic acid polymer. The second sample can be provided having a known concentration of the first nucleic acid polymer, which can be the same as or different from the concentration of the first nucleic acid polymer in the first solution. The second sample can also be provided having a known concentration of the second nucleic acid polymer. The second sample may comprise a second nucleic acid polymer, comprising a target sequence, which, depending on the sequence of the first nucleic acid polymer, for example, a probe, then hybridizes to the first nucleic acid polymer.

A coherent light beam can then be directed onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the second sample.

Positional shifts in the light bands relative to the baseline can then be detected, and the interaction of the first nucleic acid polymer with the second nucleic acid polymer can then be determined from the positional shifts of the light bands in the interference patterns. The rate of interaction between the two nucleic acid polymers can thus be monitored, thereby determining the hybridization of the nucleic acid polymers. That is, a system and method of the present invention provides a signal (i.e, positional shifts in the light bands) that is indicative of the hybridization status of the nucleic acid polymers. A plot of the hybridization status of the nucleic acid polymers can be used to determine the K_{D} of the hybridization of the nucleic acid molecules. A baseline K_{D} of completely homologous sequence binding is used as a reference point, and a K_{D} of unknown sequence identity binding that is similar to the reference point indicates tighter binding, or more complete base-pairing, indicating more sequence homology. This is referred to herein as a lower K_{D}, because the reference point of completely homologous binding is considered to be the lowest K_{D}. A higher K_{D} indicates less homologous binding and that fewer nucleotides in each nucleic acid molecule were complementarily paired with each other.

The signal data can be provided in a report. The present invention comprises reports comprising data from nucleic acid polymer interactions in a BSI device. Reports could contain, but are not limited to, affinity information (such as K_{D}), any means of quantifying the amount of target (RNA, DNA, etc) in a sample, and calibrations of the instrument in order to prepare for these types of measurements.

In an aspect, the first nucleic acid polymer and/or the second nucleic acid polymer is/are unlabeled. While the disclosed methods can be used in connection with unlabeled nucleic acid polymers, it is contemplated that the nucleic acid polymers can be optionally labeled. Labeling may occur before, during or after hybridization of the nucleic acid polymers. Such labeling can be convenient for preceding, subsequent, or simultaneous analysis by other analytical methods. Labels for nucleic acid polymers, single or double stranded formations, are known to those skilled in the art and are contemplated by the present invention.

In an aspect, the interaction is the formation of one or more covalent bonds, electrostatic bonds, hydrogen bonds, or hydrophobic interactions. In an aspect, the interaction creates a conformational change in at least one of the nucleic acid polymers. Thus, the interaction can generally be a hybridization or binding event between one or more of protein-DNA; DNA-DNA; RNA-RNA; DNA-RNA; protein-RNA; small molecule-nucleic acid polymers, single stranded nucleic acid polymers that bind intramolecularly between bases of the single stranded nucleic acid polymer. Thus, as disclosed herein, BSI can be used to detect DNA-DNA; RNA-RNA; DNA-RNA interactions. BSI can be used to detect interaction of nucleic acid polymers with PCR amplification products and/or amplicons.

Samples can be any composition comprising polynucleotides or nucleic acid polymers of interest and obtained from any bodily fluid including but not limited to, blood, urine, saliva, phlegm, gastric juices, serum, nasal secretions, fluid from mucosal surfaces, cerebrospinal fluid, pulmonary lavage, gastric lavage, bile, vaginal secretions, seminal fluid, aqueous humor, and vitreous humor, cultured cells, biopsies, or other tissue preparations. DNA or RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Probes. Part I. Theory and Nucleic Acid Preparation, P. Tijssen, ed. Elsevier (1993). In one embodiment, total RNA is isolated using the TRIzol total RNA isolation reagent (Life Technologies, Inc., Rockville, Md.) and mRNA is isolated using oligo d(T) column chromatography or glass beads. After hybridization of the isolated mRNA with known nucleic acid polymer probes, the hybridization signals obtained may reflect the complementarity of the probes and the sample nucleic acids.

In an aspect, a fluid sample can comprise at least one of a liquid or a gas. In particular aspects, a fluid sample comprises a solution of one or more nucleic acid polymers and one or more liquid solvents. A solution can be provided in an organic solvent or in water. In certain aspects, the solution can comprise man-made preparations or naturally occurring substances. In certain aspects, the solution can comprise a body fluid from a human, a mammal, an animal, insect, or a plant.

Generally, the substrate and channel can comprise any material suitable for containing and providing a sample for analysis and capable of being interrogated by the coherent light beam to generate backscattered light through reflective and refractive interaction of the light beam with a substrate/channel interface and the sample. In an aspect, the substrate and channel together comprise a capillary tube. In an aspect, wherein the substrate and channel together comprise a microfluidic device.

In an aspect, the microfluidic device comprises a polymeric substrate and an etched channel formed in the substrate for reception of a fluid sample, the channel having a cross sectional shape. In an aspect, the polymeric substrate can be selected from rigid and transparent plastics. In various further aspects, the polymeric substrate comprises one or more polymers selected from polycarbonate, polydimethylsiloxane, fluorosilicone, polytetrafluoroethylene, poly(methyl methacrylate), polyhexamethyldisilazane, polypropylene, starch-based polymers, epoxy, and acrylics.

In an aspect, the microfluidic device comprises a silica substrate and an etched channel formed in the substrate for reception of a fluid sample, the channel having a cross sectional shape, which can be substantially circular, substantially semi-circular, or substantially rectangular.

It is contemplated the substrate can comprise one or more than one channel. In an aspect, the substrate further comprises a reference channel.

The disclosed methods can provide real-time, free-solution detection of molecular interactions or real-time, determination of molecular interactions wherein one or more nucleic acid molecules are immobilized, with very low detection limits. That is, in an aspect, the invention relates to a method for real-time, free-solution determination of molecular interactions or real-time determination of molecular interactions wherein one or more nucleic acid molecules are immobilized, comprising the step of detecting the formation of one or more interaction products of two or more unlabeled, non-immobilized nucleic acid polymers, or of two or more unlabeled nucleic acid polymers wherein at least one of the nucleic acid polymers is immobilized, wherein at least one of the nucleic acid polymers is present in a sample or during a BSI measurement at a concentration of a range of about 0.01 nM to about 300 nM, of about 0.1 nM to about 300 nM, of about 1 nM to about 300 nM, of about 10.0 nM to about 300 nM, of about 100.0 nM to about 300 nM, of about 5 nM to about 200 nM, of about 0.1 nM to about 100 nM, of about 200 nM to about 300 nM, of about 100 nM to about 200 nM, at a concentration of less than about 1.0×10⁻⁵M, less than about 5.0×10⁻⁶M, less than about 1.0×10⁻⁶M, less than about 5.0×10⁻⁷M, less than about 1.0×10⁻⁷M, less than about 5.0×10⁻⁸M, less than about 1.0×10⁻⁸M, less than about 5.0×10⁻⁹M, or less than about 1.0×10⁻⁹M, less than about 5.0×10⁻¹⁰ M, less than about 1.0×10⁻¹⁰ M, less than about 5.0×10⁻¹¹M, less than about 1.0×10⁻¹¹M, less than about 5.0×10⁻¹²M, less than about 1.0×10⁻¹²M less than about 5.0×10⁻¹³M, less than about 1.0×10⁻¹³M, less than about 5.0×10⁻¹⁴M, less than about 1.0×10⁻¹⁴M, less than about 5.0×10⁻¹⁵M, or less than about 1.0×10⁻¹⁵M.

Disclosed methods herein can provide real-time, free-solution detection of molecular interactions or real-time determination of molecular interactions wherein one or more nucleic acid molecules are immobilized, with very low sample volume requirements. That is, in an aspect, the invention relates to a method for real-time, free-solution determination of molecular interactions or real-time determination of molecular interactions wherein one or more nucleic acid molecules are immobilized, comprising the step of detecting the formation of one or more interaction products of two or more unlabeled, non-immobilized nucleic acid polymers, or of two or more unlabeled nucleic acid polymers wherein at least one of the nucleic acid polymers is immobilized, wherein at least one of the nucleic acid polymers is present during the determination in a solution with a volume in the detection zone of less than about 500 nL. In various further embodiments, the sample volume can be less than about 250 nL, for example, less than about 100 nL, less than about 10 nL, less than about 1 nL, less than about 500 pL, less than about 250 pL, less than about 100 pL, less than about 50 pL, less than about 10 pL, or less than about 1 pL.

In an aspect, the disclosed methods further comprise the step of performing a chromatographic separation and/or an electrophoretic separation on the sample before, during, or after the BSI device methods steps. For example, ucleic acid polymers may be the amplicons or amplification products of one or more PCR cycles. PCR amplification reactions can be carried out according to procedures well known in the art, as discussed above (see, e.g., U.S. Pat. Nos. 4,683,195 and 4,683,202). The time and temperature of the primer extension step will depend on the polymerase, length of target nucleic acid being amplified, and primer sequence employed for the amplification. The number of reiterative steps required to sufficiently amplify the target nucleic acid will depend on the efficiency of amplification for each cycle and the starting copy number of the target nucleic acid. As is well known in the art, these parameters can be adjusted by the skilled artisan to effectuate a desired level of amplification. Those skilled in the art will understand that the present invention is not limited by variations in times, temperatures, buffer conditions, and the amplification cycles applied in the amplification process.

### Interferometric Detection Systems for Determination of Molecular Interactions of Nucleic Acid Molecules

In an aspect, the invention relates to an interferometric detection system comprising a substrate; a channel formed in the substrate for reception of a fluid sample to be analyzed; in certain aspects, a channel comprising at least an immobilized nucleic acid polymer, means for introducing a first sample comprising at least a first nucleic acid polymer; means for introducing a second or further sample(s) comprising at least a second (or other) nucleic acid polymer(s); optionally, means for mixing the first sample and the second sample; a coherent light source for generating a coherent light beam, the light source being positioned to direct the light beam onto the substrate such that the light beam is incident on the channel to thereby generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; a photodetector for receiving the backscattered light and generating one or more intensity signals that vary as a function of positional shifts of the light bands; and a signal analyzer for receiving the intensity signals, and determining therefrom, a characteristic property of the fluid sample in the channel.

In an aspect, the invention relates to a detection system comprising a microfluidic channel formed in a substrate; a solution comprising label-free nucleic acid polymers in free solution in the channel; and an interferometer that detects molecular interactions between the nucleic acid polymers in the channel. In an aspect, the invention relates to a detection system comprising a solution comprising label-free nucleic acid polymers in free solution; and a detector that detects molecular interactions between the nucleic acid polymers in the solution. The detector can be, for example, an interferometer. Likewise, detection can be, for example, by means of back-scattering interferometry. The channel can be formed by, for example, etching, by molding, by micromachining, or by photolithography. It is contemplated that the system can be used to determine one or more of an equilibrium constant, a dissociation constant, a dissociation rate, a dissociation rate constant, an association rate, and/or an association rate constant of an interaction.

In an aspect, the invention relates to a detection system comprising a microfluidic channel formed in a substrate; comprising at least an immobilized nucleic acid polymer; a solution comprising nucleic acid polymers in solution in the channel; and an interferometer that detects molecular interactions between the nucleic acid polymers in the channel. In an aspect, the invention relates to a detection system comprising immobilized nucleic acid polymers that may or may not hybridize with nucleic acid polymers provided in solution; and a detector that detects molecular interactions between the nucleic acid polymers in the channel. The detector can be, for example, an interferometer. Likewise, detection can be, for example, by means of back-scattering interferometry. The channel can be formed by, for example, etching, by molding, by micromachining, or by photolithography. It is contemplated that the system can be used to determine one or more of an equilibrium constant, a dissociation constant, a dissociation rate, a dissociation rate constant, an association rate, and/or an association rate constant of an interaction.

In various aspects, a means for introducing a sample can be any apparatus, system, or construct capable of conveying a sample into the system and/or directing a sample into or through the channel such that the light beam incident on the channel encounters at least a portion of the sample, thereby generating backscattered light through reflective and refractive interaction of the light beam with a substrate/channel interface and the sample. Examples of means for introducing a sample include an opening in a capillary tube, an injection port, a second capillary tube in fluid communication with the substrate and channel, a microfluidic channel in fluid communication with the substrate and channel, a syringe, a pipette, a chromatographic separation apparatus in fluid communication with the substrate and channel, and/or an electrophoretic separation apparatus in fluid communication with the substrate and channel.

It is understood that the means for introducing a first sample and the means for introducing a second sample or further samples can comprise the same or a different means. It is also understood that the means for introducing a first sample and the means for introducing a second sample can comprise the same type (e.g., both are injection ports) or a different type (e.g., one is a capillary tube and the other is a syringe) of means.

In various aspects, a means for mixing can be any means for apparatus, system, or construct capable of combining two samples such that the samples are in intimate contact and capable of interacting physically and/or chemically. Examples of means for mixing include a blender, a sonication apparatus, a microfluidic serpentine mixer, and a microfluidic restriction.

In an aspect, the coherent light source is a laser, for example a He/Ne laser, a VCSEL laser, or a diode laser.

In an aspect, the system can further comprise a reference channel. In an aspect, the substrate and channel together comprise a capillary tube. In an aspect, the substrate and channel together comprise a microfluidic device. In an aspect, the microfluidic device comprises a silica substrate and an etched channel formed in the substrate for reception of a fluid sample, the channel having a cross sectional shape. In an aspect, the microfluidic device comprises a glass substrate and an etched channel formed in the substrate for reception of a fluid sample, the channel having a cross sectional shape. In an aspect, the cross sectional shape is substantially rectangular, substantially circular, or generally semi-circular. In an aspect, the microfluidic device comprises a polymeric substrate and an etched channel formed in the substrate for reception of a fluid sample, the channel having a cross sectional shape. In an aspect, the polymeric substrate can be selected from rigid and transparent plastics. In various aspects, the polymeric substrate comprises one or more polymers selected from polycarbonate, polydimethylsiloxane, fluorosilicone, polytetrafluoroethylene, poly(methyl methacrylate), polyhexamethyldisilazane, polypropylene, starch-based polymers, epoxy, and acrylics.

Thus, in various aspects, the channel is formed by etching, by molding, by micromachining, or by photolithography.

A backscatter detection technique is generally disclosed in U.S. Pat. No. 5,325,170 to Bornhop, which is hereby incorporated by reference. More recently, the technique is referred to as Back-Scatter Interferometry or BSI.

The disclosed invention may comprise methods that perform interference detection in channels with ultra-small volumes and with a simple optical configuration that requires no additional optics. The BSI device is an effective universal detection system that expands the ability to sense or quantitatively detect otherwise invisible solutes, particularly those important to clinical diagnostics, proteomic, genomic and metabolomic analysis and high throughput molecular drug screening. The device's S/N ratio is not hindered by volume reduction, its probe volume and detection volume are the same, it is a non-invasive method, and is universal in nature. Thus, the device can play an important role in integrated -omics technology, drug discovery and development and diagnostic medicine. It can also allow detection of nucleic acid hybridization reactions previously not possible. Reaction kinetics can be followed in nanoliter volumes, and millidegree temperature changes can be quantified. The invention may be used in methods for cellular level analysis and bioassays and clinical diagnostic testing.

The present invention can make use of observations of the interference pattern of a BSI device and system at large angles with respect to the light path, e.g., the range of angles can include angles up to at least 10°, at least 15°, at least 20°, at least 25°, or at least 30° or can include angles of at least 35°, at least 40°, at least 45°, at least 50°, at least 55°, or at least 60°.

In an aspect, a BSI device, a system of the present invention, or a method of the present invention may comprise a thermal cycler or other device used for PCR reactions. The thermal cycler (also known as a Thermocycler, PCR Machine or DNA Amplifier) is a laboratory apparatus used to amplify segments of DNA or RNA via the polymerase chain reaction (PCR) process. A known device has a thermal block with holes where, for example, tubes, microfluidic channels, or capillary tubes holding the PCR reaction mixtures can be inserted. The cycler can then raise and lower the temperature of the block in discrete, pre-programmed steps. The apparatus can be equipped with a heated lid and a heated plate that presses against the lids of the reaction tubes. This prevents condensation of water from the reaction mixtures on the insides of the lids and makes it unnecessary to use PCR oil. The apparatus can be equipped with multiple blocks allowing several different PCR reactions to be carried out simultaneously. The apparatus can have a gradient function, which allows different temperatures in different parts of the block. The temperature of the block can be controlled by a Peltier heating element.

Data processing means can be adapted to perform an analysis which comprises one or both of: (a) the determination of the angle with respect to the light path at which there is an abrupt change in the intensity of the lighter fringes, or (b) the determination of the position of fringes of a low frequency component of the variation of intensity between the lighter and darker fringes. In an aspect, the data processing means comprises a processor programmed to determine a characteristic property of the fluid sample in the channel by performing a method comprising the steps of: computing an overlapping product of signal A and signal B generated from the detector, and assigning values to elements of a list R based on the overlapping product; summing a set of elements from R to produce a value q; multiplying a set of elements from R by an odd function; summing one or more products from the multiplying step to produce a value p; and calculating the shift between signal A and signal B as a function of p divided by q.

In an aspect, the data processing means is adapted to perform an analysis which comprises: the determination of the position of the fringes of a low frequency component of the variation of intensity between the lighter and darker fringes.

In an aspect, the data processing means is adapted to perform an analysis which comprises the determination of the angle with respect to the light path at which there is an abrupt change in the intensity of the lighter fringes.

In an aspect, the data processing means is adapted to perform an analysis which comprises the determination of the angle with respect to the light path at which there is an abrupt change in the intensity of the lighter fringes and comprises the determination of the position of the fringes of a low frequency component of the variation of intensity between the lighter and darker fringes.

In an aspect, the data processing means is adapted to perform an analysis which comprises one or both of: (a) the determination of the angle with respect to the light path at which there is an abrupt change in the intensity of the lighter fringes, or (b) the determination of the position of these fringes of a low frequency component of the variation of intensity between the lighter and darker fringes, and wherein the sample holder locates the sample between a first material defining the first interface with the sample and an material defining the second interface with the sample, which first and second materials are composed of the same substance.

As the measurement monitors a displacement of the fringe pattern, it is inherently a differential measurement, employing calibration both for the absolute level of the refractive index as well as for the differential factor. This factor describes the fringe movement corresponding to a given change in the refractive index.

The dynamic range of the BSI system may be increased by taking into account other variations of the interference pattern with changing refractive index than those previously considered. The dynamic range is increased without compromising the high differential sensitivity previously reported [Swinney, K; Markov, D; Bornhop, D. J; Review of Scientific instruments, 2000, 71, 2684 2692.]. Theoretical description of the BSI scheme has been improved to include an extended optical ray tracing model that matches the range in angular and refractive index space of the experiments, thus providing new information about the structure of the reflected light interference pattern. In contrast to the previously proposed model [Tarigan, H. J; Neill, P. Kenmore, C. K; Bornhop, D. J. Anal. Chem., 1996, 68, 1762 1770.], this model is capable of explaining all frequency components that appear in the interference pattern. Furthermore, the model has been used to predict an abrupt change in the intensity of the reflected light interference fringes, which depends uniquely on the absolute value of the refractive index of the probed sample. Moreover, this feature has been experimentally confirmed. The improved understanding of the BSI system provides two preferred approaches to an absolute measurement of the refractive index of samples, which are preferably liquids in the refractive index range between water (1.33) and glass (1.50). The first approach is based on the measurement of the depth of modulation of the interference pattern caused by variations in the Fresnel coefficients. The second approach is based on the measurement of the total internal reflection angle within the capillary or other sample container.

A BSI device and system can comprise means for controlling the temperature of the sample, e.g., a heater and/or a Peltier cooler and a temperature measuring device. As would be readily understood by one of skill, the term “back-scatter” is generally used to describe the origin of the light rays that form the interference pattern. On the basis of theoretical analysis of the origin of the interference pattern presented herein, the term “reflection” is more strictly accurate, but the phenomenon referred to by these terms is in each case the same.

In an aspect, the source of coherent light is a laser, suitably a He—Ne laser or a diode laser or VCSEL. The laser light may be coupled to the site of measurement by known wave-guiding techniques or may be conventionally directed to the measurement site by free space transmission.

The measured refractive index can be indicative of a number of properties of the sample including the presence or concentration of a solute substance, e.g. a reaction product, pressure, temperature or flow rate (e.g., by determining when a thermal perturbation in a liquid flow reaches a detector).

In an aspect, the detector is a CCD array of suitable resolution.

The present invention includes methods comprising use of an apparatus as described herein, wherein the sample holder is configured to allow a sample to flow there through and wherein the sample holder is connected to receive a separated sample from a sample separation device in which components of a mixed sample are separated, e.g., by capillary electrophoresis, capillary electrochromatography or HPLC. Accordingly, viewed from another perspective, the invention provides chromatography apparatus having a refractive index measuring unit as described herein as a detector.

More generally, the sample holder of the apparatus described above can be a flow through passage so that the contents of the channel may be continuously monitored to observe changes in the content thereof. These changes may comprise hybridization of nucleic acid polymers and the out flow from the sample holder may be diverted to a selected one of two or more outlet channels according to the measurements of RI observed in the sample holder. The sample holder may contain a stationary analytical reagent (e.g., an oligonucleotide or other selective binding agent of nucleic acids) and changes in the refractive index caused by the binding of a nucleic acid to the immobilized agent may be observed.

### Methods for Nucleic Acid Hybridization Detection

The present invention comprises compositions, methods and systems for detecting the hybridization of a nucleic acid in a sample using an interferometric detection system. The nucleic acid can be, for example, deoxyribonucleic acid (DNA) or ribonucleic acid (RNA). The nucleic acid can be genomic DNA, cDNA, plasmid DNA, or mitochondrial DNA. The nucleic acid can be genomic RNA, messenger RNA (mRNA), ribosomal RNA (sRNA), microRNA (miRNA), small nuclear RNA (snRNA), double-stranded RNA (dsRNA), or small interfering RNA (siRNA). The nucleic acid can be from the cell of a subject, organelles, or from living subjects including but not limited to human, animal, plant or insect. The nucleic acid polymer may be microbial DNA or RNA, including but not limited to a nucleic acid polymer from a virus, bacterium, yeast, mycoplasma, Archea or Protista member.

Nucleic acid polymers of the present invention may be made of for example, nucleotides, nucleotide analogs, or nucleotide substitutes. Non-limiting examples of these and other molecules are discussed herein. It is understood that for example, that a nucleic acid polymer will typically be made up of A, C, G, U and/or T nucleotides. A nucleoside/nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). As referred to herein, the nucleoside/nucleotides that form a nucleic acid polymer may be referred to as a base or bases.

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (psi), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH, F, O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

A RNA nucleic acid molecule of the present invention may comprise a locked nucleic acid (LNA) wherein the ribose moiety of the RNA molecule is modified with an extra carbon bridge connecting the 2′ oxygen and the 4′ carbon. The bridge locks the ribose in the 3′-endo (North) conformation. Such a locked ribonucleotide may be present one or more times in an RNA or DNA nucleic acid polymer sequence, for example, found every 3^{rd} base of the molecule. A structure of the modified base is shown below.

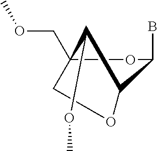

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a nucleic acid polymer, each containing a base, are referred herein as a nucleotide or a base.

It is also possible to link other types of molecules (conjugates) to nucleotides or nucleotide analogs. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

The term “hybridization” typically means a sequence driven interaction between at least two nucleic acid molecules, such as a primer or a probe and a target sequence nucleic acid polymer. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules or nucleic acid polymers are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference in its entirety and at least for material related to hybridization of nucleic acids). As used herein “stringent hybridization” for a DNA:DNA hybridization is about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid polymer is in for example, 10 or 100 or 1000 fold excess. Measurements in a BSI method or device can be performed at under conditions where both the limiting and non-limiting primer are for example, 10 fold or 100 fold or 1000 fold below their K_{D}, or where only one of the nucleic acid molecules is 10 fold or 100 fold or 1000 fold or where one or both nucleic acid molecules are above their K_{D}.

By “specifically hybridizes” is meant that a probe, nucleic acid polymer, or oligonucleotide recognizes and physically interacts (that is, base-pairs) with a substantially complementary nucleic acid (for example, a target nucleic acid) under high stringency conditions, and does not substantially base pair with other nucleic acids.

By “hybridizing under stringent conditions” or “hybridizing under highly stringent conditions” is meant that the hybridizing portion of the hybridizing nucleic acid polymer, typically comprising at least 15, e.g., 20, 25, 30, or 50 nucleotides, hybridizes to all or a portion of the provided nucleotide sequence under stringent conditions. Generally, the hybridizing portion of the hybridizing nucleic acid is at least 80%, for example, at least 90%, 95%, or 98%, identical to the sequence of or a portion of the FCGR2B promoter nucleic acid of the invention, or its complement. Hybridizing nucleic acids of the invention can be used, for example, as a cloning probe, a primer (e.g., for PCR), a diagnostic probe, or an antisense probe. Hybridization of the oligonucleotide probe or nucleic acid polymer to a nucleic acid sample typically is performed under stringent conditions. Nucleic acid duplex or hybrid stability may be expressed as the melting temperature or Tm, which is the temperature at which a probe dissociates from a target DNA. This melting temperature may be used to define the required stringency conditions. If sequences are to be identified that are related and substantially identical to the probe, rather than identical, then it may be useful to establish the lowest temperature at which only homologous hybridization occurs with a particular concentration of salt (e.g., SSC or SSPE). Assuming that a 1% mismatch results in a 1° C. decrease in the Tm, the temperature of the final wash in the hybridization reaction is reduced accordingly (for example, if sequence having >95% identity with the probe are sought, the final wash temperature is decreased by 5° C.). In practice, the change in Tm can be between 0.5° C. and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the target nucleic acid. Additional guidance regarding such conditions is readily available in the art, for example, in Sambrook et al., 1989, Molecular Cloning, A Laboratory Manual, Cold Spring Harbor Press, NY; and Ausubel et al. (eds.), 1995, Current Protocols in Molecular Biology, (John Wiley & Sons, NY) at Unit 2.10.

By “probe”, “oligonucleotide probe” or grammatical equivalents as used herein is meant a nucleic acid polymer or molecule that will hybridize to some portion of a target or second nucleic acid polymer or oligonucleotide molecule. Probes of the present invention may be designed to be substantially complementary to a target sequence such that hybridization occurs between the target sequence and the probe molecules. As used herein, mismatch or mismatching bases means that one or more nucleotide bases in a first nucleic acid polymer do not hybridize with one or more corresponding bases in a second nucleic acid polymer. For example, in the first nucleic acid polymer at base number 10 in the sequence an A is present, and in the second nucleic acid polymer at base number 10 a G is present. The two nucleic acid polymers would hybridize at all base pairs of the two sequences except at base number 10, because A and G do not hybridize with each other. The base pairs at the location in the nucleic acid polymers of base number 10 are mismatched. Probes of the present invention may have a known sequence, and may be used in methods so that hybridization reactions with target sequences result in a mismatching of bases of the probe with the target sequence nucleic acid polymer so that the mismatched bases occur at or near one end of the nucleic acid polymers or may occur at a site internal to the ends of the nucleic acid polymers.

The present invention comprises a probe nucleic acid polymer having a sequence that is complementary to a target sequence at all bases of the nucleic acid polymer (nucleotide base—for example, A,C,T,G,U) except for 1 nucleotide base which is a mismatch for 1 base in the target sequence located in the corresponding position in the target sequence. The present invention comprises a probe nucleic acid polymer having a sequence that is complementary to a target sequence at all bases of the nucleic acid polymer, except for at least 1 nucleotide base which is a mismatch for at least 1 base in the target sequence located in the corresponding position in the target sequence; probes that are complementary except for at least 2 nucleotide bases which are a mismatch for at least 2 bases in the target sequence located in the corresponding positions in the target sequence; probes that are complementary except for at least 3 nucleotide bases which are a mismatch for at least 3 bases in the target sequence located in the corresponding positions in the target sequence; probes that are complementary except for at least 4 nucleotide bases which are a mismatch for at least 4 bases in the target sequence located in the corresponding positions in the target sequence; probes that are complementary except for at least 5 nucleotide bases which are a mismatch for at least 5 bases in the target sequence located in the corresponding positions in the target sequence; probes that are complementary except for at least 10 nucleotide bases which are a mismatch for at least 10 bases in the target sequence located in the corresponding positions in the target sequence; probes that are complementary except for at least 12 nucleotide bases which are a mismatch for at least 12 bases in the target sequence located in the corresponding positions in the target sequence; probes that are complementary except for at least 20 nucleotide bases which are a mismatch for at least 20 bases in the target sequence located in the corresponding positions in the target sequence; probes that are complementary except for at least 25 nucleotide bases which are a mismatch for at least 25 bases in the target sequence located in the corresponding positions in the target sequence; probes that are complementary except for at least 30 nucleotide bases which are a mismatch for at least 30 bases in the target sequence located in the corresponding positions in the target sequence. The present invention comprises a probe nucleic acid polymer having a sequence that is complementary to a target sequence at all bases of the nucleic acid polymer, except for a range of from about 1 nucleotide to about 100 nucleotides, from about 1 nucleotide to about 10 nucleotides, from about 10 nucleotide to about 100 nucleotides, from about 1 nucleotide to about 5 nucleotides, from about 1 nucleotide to about 3 nucleotides, from about 1 nucleotide to about 2 nucleotides, from about 5 nucleotide to about 50 nucleotides, from about 1 nucleotide to about 20 nucleotides, from about 1 nucleotide to about 15 nucleotides, from about 1 nucleotide to about 40 nucleotides, from about 5 nucleotide to about 10 nucleotides, and all ranges therebetween.

The mismatch bases may be contiguous with one another, or may be located in several locations in the sequence, or may be randomly located in the sequence, or may be individually located throughout the sequence. The mismatch base or bases may be located at the terminal end or ends of a sequence, or may be at a location internal to the terminal end or ends, and may be at least 1 nucleotide from an end, at least 2 nucleotides from an end, at least 3 nucleotides from an end, at least 5 nucleotides from an end, at least 10 nucleotides from an end, at least 15 nucleotides from an end, at least 20 nucleotides from an end, at least 20 nucleotides from an end, at least 25 nucleotides from an end, at least 30 nucleotides from an end, at least 35 nucleotides from an end, at least 40 nucleotides from an end, at least 50 nucleotides from an end, or at least 100 nucleotides from an end. The present invention comprises probes having a sequence comprising one or more bases, a mismatch base or bases, that are not complementary to bases in a target nucleic acid polymer sequence, wherein such mismatch bases are located at sites in the probe sequence that correspond to sites in target sequences, and wherein the probe sequence and the target sequence are substantially complementary in the rest of the respective sequences. The present invention comprises compositions, reaction mixtures and kits comprising probes disclosed herein.

As disclosed herein, the present invention comprises methods of detecting mismatch bases in hybridized nucleic acid polymers using BSI methods and devices. Such methods comprise BSI detection of mismatch bases in hybridized nucleic acid polymers where the mismatch site is at a terminal end of the duplex formed by hybridization, detection of mismatch bases in hybridized nucleic acid polymers where the mismatch is at a location internal to a terminal end of the duplex formed by hybridization, Such hybridization reactions may occur in free solution conditions where a first and a second nucleic acid polymers are non-immobilized, or may occur in immobilized reaction conditions wherein at least a first nucleic acid polymer is immobilized on a surface.

The present invention comprises methods for detecting the location of a sequence mutation (or change of one or more bases of the sequence) in a target nucleic acid sequence. Such mutations may be SNP, single nucleotide polymorphisms or may comprise 2 or more nucleotides. For example, a method comprises free-solution detection of molecular interactions by BSI comprising providing a BSI device, such as a substrate having a channel formed therein for reception of a fluid sample to be analyzed; introducing a composition comprising a first non-immobilized nucleic acid polymer probe into the channel, wherein the probe sequence is known; optionally establishing a baseline interferometric response by directing a coherent light beam onto the substrate such that the light beam is incident on the channel to generate backscattered light through reflective and refractive interaction of the light beam with a substrate/channel interface and the probe composition, the backscattered light comprising interference fringe patterns including a plurality of spaced light bands whose positions shift in response to changes in the refractive index of the first composition; introducing a second composition comprising a second non-immobilized nucleic acid polymer composition comprising a target sequence into the channel, wherein the first nucleic acid polymer interacts with the second nucleic acid polymer to form one or more interaction products, such as hybridized duplex nucleic acid polymers; directing a coherent light beam onto the substrate such that the light beam is incident on the channel to generate backscattered light through reflective and refractive interaction of the light beam with a substrate/channel interface and the compositions, the backscattered light comprising interference fringe patterns including a plurality of spaced light bands whose positions shift in response to changes in the refractive index of the compositions and interaction products in the channel; detecting positional shifts in the light bands relative to the baseline; and determining the formation of the one or more interaction products of the first nucleic acid polymer with the second nucleic acid polymer from the positional shifts of the light bands in the interference patterns.

The present invention comprises methods for detecting the location of a sequence mutation (or change of one or more bases of the sequence) in a target nucleic acid sequence. Such mutations may be SNP, single nucleotide polymorphisms or may comprise 2 or more nucleotides. For example, a method comprises detection of molecular interactions by BSI comprising providing a BSI device, such as a substrate having a channel formed therein for reception of a fluid sample to be analyzed; immobilizing at least a first nucleic acid polymer probe in the channel, wherein the probe sequence is known; or alternatively, providing a BSI device having at least a first nucleic acid polymer probe immobilized in the channel, wherein the probe sequence is known; optionally establishing a baseline interferometric response by directing a coherent light beam onto the substrate such that the light beam is incident on the channel to generate backscattered light through reflective and refractive interaction of the light beam with a substrate/channel interface and the probe composition, the backscattered light comprising interference fringe patterns including a plurality of spaced light bands whose positions shift in response to changes in the refractive index of the first composition; introducing a composition comprising at least a non-immobilized nucleic acid polymer comprising a target sequence into the channel, wherein the immobilized nucleic acid polymers interact with the non-immobilized nucleic acid polymers to form one or more interaction products or hybridization products, such as hybridized duplex nucleic acid polymers; directing a coherent light beam onto the substrate such that the light beam is incident on the channel to generate backscattered light through reflective and refractive interaction of the light beam with a substrate/channel interface and the contents of the channel, the backscattered light comprising interference fringe patterns including a plurality of spaced light bands which positions shift in response to changes in the refractive index of the compositions and interaction products in the channel; detecting positional shifts in the light bands relative to the baseline; and determining the formation of the one or more interaction products of the immobilized nucleic acid polymer with the non-immobilized nucleic acid polymer from the positional shifts of the light bands in the interference patterns.

A known probe sequence of the present invention may be the complement of, or is identical to, a normal or non-mutated sequence for target sequence, such that the probe and the target sequence form substantially hybridized duplex nucleic acid polymers, or the probe, having the normal or non-mutated sequence will have mismatched bases when hybridizing with a target sequence nucleic acid polymer that has mutated nucleotides or nucleotides that are different from those of the normal or non-mutated sequence. A known probe sequence of the present invention may be a mutated sequence or a sequence that has known SNPs or 2 or more bases that differ from the normal or non-mutated sequence. A known probe sequence of the present invention may be a mutated sequence or a sequence that differs from the normal or non-mutated sequence by 1 or more bases. Such a mutated sequence probe is the complement of, or is identical to, a normal or non-mutated sequence for a target sequence except for the SNP or different sequence bases, such that the probe and a normal, non-mutated target sequence do not form substantially hybridized duplex nucleic acid polymers because there is one or more mismatch bases, or the probe, having the mutated sequence will substantially hybridize with a target sequence nucleic acid polymer that has mutated nucleotides or nucleotides that are different from those of the normal or non-mutated sequence. Hybridization products formed by the probe and target sequence interaction are detected using a BSI device. BSI device measurements can distinguish between hybridization products where there is substantially complete hybridization, with substantially 100% base matching between the probe and the target sequence; hybridization products where there is 1 or more mismatch bases located on a terminal end of the hybridized duplex nucleic acid polymers; and hybridization products where there is 1 or more mismatch bases located at a position internal from a terminal end of the hybridized duplex nucleic acid polymers. A target sequence nucleic acid polymer may be single or double stranded, and a double-stranded nucleic acid polymer may be denatured prior to, or during, a hybridization step.

The positional shifts of the light bands in the interference patterns can be used to determine the location of the mismatch bases, thus providing the location of the sequence change in the target sequence without the need to sequence the target sequence. As disclosed herein, BSI detection can readily distinguish between mismatch bases found on the ends of hybridized duplex nucleic acid polymers and mismatch bases found internally from the ends of hybridized duplex nucleic acid polymers. BSI detection can be used to determine the free solution and surface immobilized K_{D} for mismatch bases found on the ends of hybridized duplex nucleic acid polymers and mismatch bases found internally from the ends of hybridized duplex nucleic acid polymers. By knowing the sequence of one or more probes and hybridizing the one or more probes with a target sequence, determining the K_{D} of the hybridized products, one can determine the location of mismatch bases in the hybridized products, and the location of a mutation in the target sequence without the need to sequence the target sequence.

A method of the present invention for determining the location of a SNP or one or more bases in a sequence that differ from a reference sequence, such as a normal or mutated sequence, comprises a) hybridizing a probe having a known sequence with a target sequence, b) using a BSI device to direct a coherent light beam to generate backscattered light comprising interference fringe patterns including a plurality of spaced light bands which positions shift in response to changes in the refractive index of the compositions and interaction products in the channel; c) detecting positional shifts in the light bands relative to the baseline; and d) determining the formation of one or more interaction products from the positional shifts of the light bands in the interference patterns. Steps a)-d) can be repeated using derivative probes. A derivative probe is a probe having a sequence that is similar the sequences of other derivative probes, but the sequence of each probe is changed so that mismatch bases in hybridized products will occur at a site in a particular derivative probe that is different from that of another derivative probe. For example derivative probes may have a known sequence wherein potential mismatch or mismatch bases of the probe sequence are located at the same sequence sites but derivative probe nucleic acid polymers comprise different portions of the sequence so that the mismatch or potential mismatch bases are located at a terminus, internal from a terminus, or at the center of a probe nucleic acid polymer. For example, a normal sequence is 5′ATCGGGTTAACCT 3′, (SEQ ID No. 1) and a sequence that is complementary to that sequence is 5′AGGTTAACCCGAT 3′(SEQ ID No. 2). A first derivative probe with a mismatch sequence is 5′AGGTTAACTTGAT 3′ (SEQ ID No. 3), and other derivative probes, or a set of probes, may comprise the first derivative probe and other. derivatives probes having sequences of 5′AGGTTAACCGTT3′ (terminus mismatch) (SEQ ID No. 4), 5′AGGTTAACTTGAT 3′ (internal mismatch) (SEQ ID No. 5) and 5′ AGGTTAACTTGAT 3′ (central mismatch) (SEQ ID No. 6). Alternatively, probes may be designed so that the mismatch is located at different sites in the sequence. For example, a normal sequence is 5′ATCGGGT 3′, and sequence that is complementary to that sequence is 5′ACCCGAT3′. A derivative probe that would form a mismatch on a terminus is 5′GTCCGAT3′ (mismatch in bold). A derivative probe that would form a mismatch internally from a terminus is 5′ACCTTAT 3′ (mismatch in bold). A set of probes providing mismatch bases in different locations of the probe, terminus, internally or central, may be referred to as a derivative probe set and may be used in a series of hybridization product reactions with a target sequence to distinguish where a mutation or altered sequence is located in a target sequence, without the need to sequence or perform PCR with the target sequence.

A method may comprise providing one or more nucleic acid polymer probes that each comprise at least 3, 4 5, 6, 7, 8, 9, 10, 11, 12, 13, 14, 15, 16, 17, 18, 19, 20, 25, 30 or more nucleotides comprising a sequence having at least about 70%, at least about 75%, at least about 80%, at least about 85%, at least about 90%, at least about 95%, at least about 96%, at least about 97%, at least about 98%, at least about 99%, or at least about 100% sequence identity to the target nucleic acid polymer or complement thereof.

The present invention comprises reagents, kits, reaction mixtures and compositions comprising probes that are capable of interacting with a target nucleic acid polymer sequence. The nucleic acid polymers may be nucleic acid polymers that result from reactions that include, but are not limited to, PCR, DNA sequencing, DNA extension, DNA polymerization, RNA transcription, or reverse transcription. Typically the probes of the present invention hybridize with a gene or region of the gene or hybridize with the complement of a gene or complement of a region of a target gene.

The size of the probes for interaction with the target gene in certain embodiments can be any size. A typical probe may be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Complementarity of the probes to the target nucleic acid need not be perfect. Thus, by “complementary” or “substantially complementary” herein is meant that the probes are sufficiently complementary to the target sequences to hybridize under normal reaction conditions. Deviations from perfect complementary are permissible so long as deviations are not sufficient to completely preclude hybridization. However, if the number of alterations or mutations is sufficient such that no hybridization can occur under the least stringent of hybridization conditions, the sequence is not a complementary target sequence. The methods of the present invention can detect mismatched DNA hybridization for as few as a complementary pair, or two mismatched bases, one on each nucleic acid polymer.

The nucleic acid polymers as described herein may be single stranded or double stranded, as specified, or contain portions of both double stranded or single stranded sequence. The nucleic acid polymer may be DNA, RNA, or hybrid, where the nucleic acid polymer contains any combination of deoxyribo- and ribonucleotides, and any combination of bases, including uracil, adenine, thymine, cytosine, guanine, xanthine hypoxanthine, isocytosine, isoguanine, inosine, etc.

The size of the probe nucleic acid polymer may vary, as will be appreciated by those in the art, in general varying from 5 to 500 nucleotides in length. For example, with probes of between 10 and 100 nucleotides, between 12 and 75 nucleotides, and from 15 to 50 nucleotides can be used, depending on the use, or required specificity.

The stability of the hybridized nucleic acid polymers, especially the thermal melting temperature (Tm), may be determined by methods well known in the art. These include, but are not limited to, nearest-neighbor thermodynamic calculations (Breslauer, T. et al., Proc. Natl. Acad. Sci. USA 83:8893-97 (1986); Wetmur, J. G., Crit. Rev. Biochem. Mol. Biol. 26:227-59 (1991); Rychlik, W. et al., J. NIH Res. 6:78 (1994)), Wallace Rule estimations (Suggs, S. V. et al “Use of Synthetic oligodeoxyribonucleotides for the isolation of specific cloned DNA sequences,” Developmental biology using purified genes, D. B. Brown, ed., pp 683-693, Academic Press, New York (1981), and Tm estimations based on Bolton and McCarthy (see Baldino, F. J. et al., Methods Enzymol. 168: 761-77 (1989); Sambrook, J. et al., Molecular Cloning: A Laboratory Manual, Chapter 10, Cold Spring Harbor Laboratory Press, Cold Spring Harbor, N.Y., (2001)). All references are hereby expressly incorporated by reference. The effect of various parameters, including, but not limited to, ionic strength, probe length, G/C content, and mismatches are taken into consideration when assessing hybridization product stability. Consideration of these factors is well known to those skilled in the art.

In hybridizing the probes, also referred to herein as a first nucleic acid polymer, to the target nucleic acid polymers, the assays are generally done under stringency conditions that allow formation of hybridization products in the presence of target sequence nucleic acid polymers, referred to herein as a second nucleic acid polymer. Those skilled in the art can alter the parameters of temperature, salt concentration, pH, organic solvent, chaotropic agents, or other variables to control the stringency of hybridization.

The present invention comprises compositions and methods comprising the use of an interferometric detection device and system to detect nucleic acid polymer hybridization. An aspect of the present invention comprises detection of sequence mismatch between a probe nucleic acid polymer, also referred to herein as a first nucleic acid polymer, and a target nucleic acid polymer, also referred to herein as a second nucleic acid polymer. Methods for easily and reliably detecting point mutations without prior knowledge of the exact location of the mutations, have wide application in diagnosis and treatment of genetic diseases and cancers, as well as use in genetic counseling. These methods also are of great benefit in the analysis of a variety of human genetic diseases and in establishing human genetic linkage maps.

In many genetic diseases, the causative mutations are scattered over a large number of sites. For example in retinoblastoma, 30% of the cases are the result of widely scattered new mutations (Yandell et al., 1989). In addition, accumulation of point mutations in a variety of genes, such as, for example, p53, ras, and other “protooncogenes” are thought to play fundamental roles in the multi-step process of transformation of normal cells to the malignant state. The ability to detect these mutations is important both for genetic counseling and for early clinical intervention. Improved efficiency and reliability in methods of detecting point mutations should lead to a better understanding of the mechanisms of carcinogenesis and to improved treatment and prognosis for a variety of cancers. The ideal screening method would quickly, inexpensively, and reliably detect all types of widely dispersed point mutations, insertions/deletions, and translocations in genomic DNA, cDNA, or RNA samples depending on the specific situation. The present invention comprises methods of hybridization of at least a probe nucleic acid polymer having a known sequence (for example, a first nucleic acid polymer(s)) with at least a target nucleic acid polymer (for example, second nucleic acid polymer(s)) and detecting the hybridization of the nucleic acid polymers.

A number of different methods have been used to detect single-base mutations. These methods include denaturing gradient gel electrophoresis, restriction enzyme polymorphism analysis, chemical mismatch methods and others (see Cotton, 1989, for a review of single-base mutation detection methods). Recently, SSCP (single-strand conformation polymorphism) analysis and the closely related heteroduplex analysis methods have come into use for screening for single-base mutations (Orita et al., 1989; Keen et al., 1991). In these methods, the mobility of PCR-amplified test DNA from clinical specimens is compared with the mobility of DNA amplified from normal sources by direct electrophoresis of samples in adjacent lanes of native polyacrylamide or other types of matrix gels. Single-base mutations often alter the secondary structure of the molecule sufficiently to cause slight mobility differences between the normal and mutant PCR products after prolonged electrophoresis.

Not all mutations result in detectable shifts in mobility. Since mobility differences are generally quite small, analysis of genes in the heterozygous state is compromised. SSCP and related techniques do not provide information on the position of the mutation within the DNA fragment being analyzed. The present invention comprises methods that can distinguish between mismatched nucleotide hybridization that is found at the end of the probe-target nucleic acid polymer hybrid and internally located mismatched sequences in the probe-target nucleic acid polymer.

Direct sequencing of PCR products and ribonuclease protection assay (RPA) are techniques used for detection of dispersed single-base mutations, though there are draw-backs to both techniques. As taught by the present invention, mismatch detection can be used to detect point mutations. Nucleic acid polymer probes can be used to detect point or other mutation by mismatch binding of one or more probes with known sequences with target nucleic acid polymers, and the hybridization of a probe with a known sequence that forms a mismatched hybridization product with a sample nucleic acid polymer can be detected using BSI devices and systems, as disclosed herein.

The disclosed compositions and methods can be used to detect specific microbes in patient, environmental, or other samples using primers specific for each microbe desired to be detected. The disclosed compositions and methods can also be used to look for biomarkers in mRNA transcriptional screening, or genotyping. The disclosed compositions and methods can also be used for allelotyping, SNP, and STTR analysis. This ‘diagnostic’ method would be more rapid than current techniques and advantageous because no signaling label would be necessary.

The detection of specific DNA or RNA sequences could be used for the diagnosis of infectious diseases, as well as human physiological diseases (i.e. any human disease where a specific nucleic acid sequence is a marker for the disease, including gene analysis involving the search for mutations). Obviously if the proposed methodology can be used for human diseases, it could be used in veterinary and agricultural applications as well as in human health.

Clinical samples of blood, saliva, urine, stool, serum, or sputum can be extracted from patients. Following extraction, low speed spins can be performed to pellet intact eukaryotic cells. Alternatively, enzymatic eukaryotic cell lysis can be carried out to enable detection of intracellular microbes (viruses, intracellular bacteria, etc). Following centrifugation, the remaining supernatant can be removed and subjected to commercially available chromatography columns that remove abundant serum proteins that may interfere with PCR such as albumin and transferrin. Next, samples can be subjected to one of a variety of techniques that promote bacterial lysis (sonication, mechanical disruption, enzymatic lysis, boiling, etc). Samples containing released microbial DNA can be evaluated using the disclosed compositions and methods to detect the presence of pathogens.

The significant improvement in sensitivity in using BSI methods and devices over currently available PCR detection systems allows for the detection in a sample of a very small number of pathogen-associated DNA or RNA nucleic acid molecules. To ensure a wide range of organisms are sampled, a variety of probes encompassing clinical relevant pathogens (and associated virulence determinants) can be included in distinct microfluidics chambers for multiplexing. Probes can be chosen based on the clinical symptoms observed in the patient and tailored based, on known epidemiologic patterns. For instance, a panel of probes can be produced that are specific for blood borne pathogens or pathogens that are often associated with chronic pulmonary diseases. The label-free nature of the disclosed compositions and methods allows comprehensive panels of probes to be produced and used without significant cost to the user.

Furthermore, by using additional probes designed for each organism, the disclosed compositions and methods can be used for typing and identifying subspecies of organisms, as well as determining unusual metabolic characteristics of those subtypes such as antibiotic resistance profiles, presence of plasmids, etc. Considering the requirement for multiplexed reactions (and hence multiple probes) aimed at identifying numerous pathogens and their associated antibiotic resistance profiles, the application of the disclosed compositions and methods to pathogen detection has the potential to result in cost savings in excess of hundreds of dollars per reaction. Finally, the majority of existing real time PCR applications require primer pairs or probes that are separated by a relatively short distance (<300 base pairs). This limitation does not apply to BSI since the distance between two probes (and hence the size of the amplicon) is not a limiting factor for detection of PCR products using BSI.

Methods of the present invention may be used for diagnostics. A diagnostic method comprising a BSI device may be portable and easily used in hospital, clinic, or in the field applications. Personalized medicine applications are contemplated by the present invention, for example, by categorizing patients into genetically definable classes that, for example, have similar drug effects (as, for example, human races, or any population group carrying a particular set of genes). Adverse drug reactions (ADRs) are a significant cause of morbidity and mortality. The majority of these cases can be related to the alterations in expression of clinical phenotype that is strongly influenced by environmental variables. Application of the present invention methods for nucleic acid polymer hybridization detection, optionally combined with other molecular techniques, make possible the monitoring of both therapeutic intervention, and individual responses to drugs.

The present invention methods, systems and compositions may be used for the detection, diagnosis and prognosis of cancer and the effectiveness of cancer treatments for patients. Cancer may arise from the accumulation of inherited polymorphism (SNPs) and mutation and/or sporadic somatic polymorphism (i.e. non-germline polymorphism) in cell cycle, DNA repair, and growth signaling genes. Despite advances in diagnostic imaging technology, surgical management, and therapeutic modalities, cancer remains a major cause of mortality worldwide. A reliable method to monitor progress of cancer therapeutic agents can be of immense use. The present invention comprises a sensitive method to quantify specific DNA using detection of DNA hybridization in a BSI device. Most of the commonly occurring cancers can be detected by measuring marker gene expressions or by using probes. A multigene panel for most common malignant diseases (carcinoma of bladder, breast cancer, colorectal cancer, endometrial carcinoma) can increase the accuracy of diagnosis which may be important as each of these cancers have excellent prognosis if diagnosed at early stage.

The present invention comprises methods for detecting nucleic acid polymer hybridization that is useful for detecting or quantifying viruses, for example from viral infected specimens. With probes specific for a particular target gene, that gene or genetic variants of the gene, which would form mismatch hybridization products, can be detected using BSI devices and systems. For example, the presence or absence of HSV1 and HSV2 can be confirmed with the methods and compositions of the present invention. Genetic changes in such viruses can also be tracked by the hybridization methods of the present invention. For example, when a viral sequence mutates, the mismatch hybridization between a probe and the mutated viral sequence can be detected by the present invention. Those skilled in the art can use the methods and composition disclosed herein to detect viral sequences including but not limited to, genital herpes, which is the most common sexually transmitted disease (STD) around the world, HBV, HCV, and other hepatitis viruses, varicella-zoster virus and other human, animal, plant and insect pathogens, or commensals. The present invention may be used for following the interactions between virus and the host, and can be used to study the efficacy of antiviral compounds or to determine the chronic conditions. Co-infections may be detected using the methods of the present invention as well as detection of viral genotype differentiation.

The present invention can be used for identifying bacteria, which can aid in identifying the correct treatments and pharmaceutical agents necessary to treat a bacterial infection. Traditionally, initial antibiotic therapy was based on identifying the Gram stain classification of the bacteria in a sample. High variability that existed in identification of bacterial pathogens by mere observations was enhanced by use of conventional PCR-based methods. The present invention comprises hybridization probes that can allow for a fast detection of low amounts of bacterial DNA and a correct Gram stain classification. A quicker conformation of the pathogen will facilitate early prescription of appropriate antibiotics.

Mycobacterium species of common interest include Mycobacterium tuberculosis, M. avium, M. bovis, M. bovis BCG, M. abscessus, M. chelonae and M. ulcerans can be detected using specific probes in the methods of the present invention. Detection of resistant isolates with mutant genes isoniazid (katG), rifampin (rpoB) and ethambutol (embB) from culture or clinical specimens can be detected using methods and systems of the present invention.

Bacteria represent the potential agents for biological warfare. The present invention, for example in a portable format, can be used to detect the presence of bacteria in samples from the field, while in the field.

The methods and compositions of the present invention can be used to detect, classify, and monitor the efficacy of treatments for fungi. Major fungi causing infections in humans are Aspergillus species (A. fumigatus, A. flavus, A. niger, A. nidulans, A. terreus, A. versicolor), Candida species (C. albicans, C. dub-liniensis), and Pneumocystis jiroveci. The conventional methods developed for detection of these infectious fungi are culturing, histopathology/phenotypic assays/biochemicals/microscopy, conventional PCR, nucleic acid probe, CFU quantification, broth dilution and staining followed by microscopic observations. The methods of the present invention for detecting and measuring the presence of nucleic acid polymers hybridization can be used for fungal nucleic acid polymers. Assays can be developed for other fungi such as Coccidioides sp., Conidiobolus sp., Cryptococcus sp., Histoplasma sp., Pneumocystis sp., Paracoccidioides sp., and Stachybotrys sp.

The methods and compositions of the present invention can be used for detection of protozoa, with clinical applications for detecting amoebic dysentery, chagas' disease, cutaneous and visceral leishmaniasis, giardiasis, Cyclospora cayeta-nensis causing prolonged gastroenteritis, toxoplas-mosis in the amniotic fluid of pregnant women, and in immuno-compromised patients. Protozoans cause several diseases, which are endemic in large parts of the world. Further genome sequencing efforts are requested as many parasitologists work on organisms whose genomes have been only partially sequenced and where little, if any, annotation is available. Such methods are useful for mycoplasma detection and monitoring.

Methods, systems and compositions of the present invention are applicable for detecting and monitoring food safety. A standardized method for the detection of food-borne pathogens should optimally fulfill various criteria such as analytical and diagnostic accuracy, high detection probability, high robustness (including an internal amplification control [IAC]), low carryover contamination, and acceptance by easily accessible and user-friendly protocols for its application and interpretation. The methods of the present invention can meet all these criteria. Pathogens such as enteroinvasive Escherichia coli, enteropatho-genic E. coli, enterohemorrhagic E. coli, enterotoxigenic E. coli, enteroaggregative E. coli, Salmonella. spp., Shigella spp., Yersinia enterocolitica, Yersinia pseudotuberculosis, Campylobacter jejuni, Vibrio cholerae, Vibrio parahaemo-lyticus, Vibrio vulnificus, Aeromonas spp., Staphylococcus aureus, Clostridium perfringens, Bacillus cereus, Plesio-monas shigelloides and Providencia alcalifaciens can be detected using probes of a known sequence in the methods described herein. Further, detection assays for Clostridium botulinum applicable to both purified DNA and crude DNA extracted from cultures and enrichment broths as well as DNA extracted directly from clinical and food specimens can be used.

Advanced technologies for DNA analysis using short tandem repeats (STR) sequences has brought about a revolution in forensic investigations. One of the most common methods used is PCR, which allows accurate genotype information from samples. Methods of the present invention may be useful in monitoring the hyper-variable region (HV1) using probes. Forensic samples are often contaminated with PCR inhibitors and DNA extraction methods fail to exclude the contaminants. The present invention is not limited to PCR and can overcome such sample issues.

The present invention comprises methods and compositions which are applicable in most, if not all, methods that currently comprise use of PCR. For example, and not as a limiting list, environmental applications, plant-host interactions, detection of mobility of genetic elements, study of gene expression patterns, species identification, and allelic discrimination such as small insertions or deletions (such as the three nucleotide deletion in the common cystic fibrosis (CFTR) allele, F508del), to name a few applications.

The present invention may also comprise use of PCR to provide sample nucleic acid polymers. With the sensitivity of the present invention, the nucleic acid polymers may be sufficient from only a few cycles of PCR. With PCR it is possible to amplify a single or few copies of a piece of DNA across several orders of magnitude, generating millions or more copies of the DNA piece. PCR can be extensively modified to perform a wide array of genetic manipulations. PCR, in its many modifications, are well known to those skilled in the art. PCR is commonly carried out in a reaction volume of 10-200 μl in small reaction tubes (0.2-0.5 ml volumes) in a thermal cycler. An advantage of the disclosed compositions and methods is that the sample size can be much smaller than conventional techniques. For example, the sample size can be less than about 500 nL, less than about 250 nL, less than about 100 nL, less than about 10 nL, less than about 1 nL, less than about 500 pL, less than about 250 pL, less than about 100 pL, or less than about 10 pL. An advantage of the disclosed compositions and methods is that the concentration of the target nucleic acid in the sample can be much smaller than conventional techniques. For example, the nucleic acid in the sample can be at a concentration of a range of about 0.01 nM to about 300 nM, of about 0.1 nM to about 300 nM, of about 1 nM to about 300 nM, of about 10.0 nM to about 300 nM, of about 100.0 nM to about 300 nM, of about 5 nM to about 200 nM, of about 0.1 nM to about 100 nM, of about 200 nM to about 300 nM, of about 100 nM to about 200 nM, at a concentration of less than about 1.0×10⁻⁵M, less than about 5.0×10⁻⁶M, less than about 1.0×10⁻⁶M, less than about 5.0×10⁻⁷M, less than about 1.0×10⁻⁷M, less than about 5.0×10⁻⁸M, less than about 1.0×10⁻⁸M, less than about 5.0×10⁻⁹M, or less than about 1.0×10⁻⁹M, less than about 5.0×10⁻¹⁰ M, less than about 1.0×10⁻¹⁰ M, less than about 5.0×10⁻¹¹M, less than about 1.0×10⁻¹¹M, less than about 5.0×10⁻¹²M, less than about 1.0×10⁻¹²M, less than about 5.0×10⁻¹³M, less than about 1.0×10⁻¹³M, less than about 5.0×10⁻¹⁴M, less than about 1.0×10⁻¹⁴M, less than about 5.0×10⁻¹⁵M, or less than about 1.0×10⁻¹⁵M.

The disclosed compositions and methods can be used in combination with variations of the traditional PCR technique. Some variations on the basic PCR technique include Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification (MLPA), Multiplex-PCR, Nested PCR, Overlap-extension PCR, Quantitative PCR (Q-PCR), RT-PCR, Solid Phase PCR, TAIL-PCR, Touchdown PCR, PAN-AC, and Universal Fast Walking. Thus, interferometric detection can be used in conjunction with these or other nucleic acid detection techniques.

Disclosed herein are methods comprising detecting a target nucleic acid from a polymerase chain reaction (PCR) by back-scattering interferometry. In some aspects, the PCR employs label-free oligonucleotides and dNTPs. In some aspects, the PCR is isothermal. For example, the PCR can employ helicase. In some aspects, the target nucleic acid is present in a concentration of less than about 5.0×10-7M. In some aspects, the target nucleic acid is present in a concentration of less than about 5.0×10-9M. In some aspects, the nucleic acid polymers generated in PCR are detected by back-scattering interferometry after 1, 2, 3, 4, 5, 6, 7, 8, 9, or 10 cycles.

In some aspects, the nucleic acid polymer hybridization occurs in a sample in a microfluidic channel in a substrate and back-scattering interferometry comprises directing a coherent light beam onto the substrate such that the light beam is incident on the channel to generate backscattered light through reflective and refractive interaction of the light beam with a substrate channel interface and the sample, the backscattered light comprising interference fringe patterns including a plurality of spaced light bands whose positions shift in response to changes in the refractive index of the fluid sample.

A disclosed PCR method comprises a) providing a BSI device, such as a substrate having a channel formed therein for reception of a fluid sample to be analyzed; b) introducing a sample from a polymerase chain reaction (PCR) after 1, 2, 3, 4, 5, 6, 7, 8, 9, or 10 or more cycles into the channel; c) providing at least a probe nucleic acid polymer with a known sequence that may or may not be immobilized in the channel, d) providing hybridization conditions so that the probe(s) can hybridize with its complement nucleic acid polymer; e) directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; and d) detecting positional shifts in the light bands wherein the positional shifts in the light bands detect hybridization of the probe to the amplified DNA (or with mutations or sequences, etc), and optionally determining the K_{D} of the hybridization interactions

In some aspects, a phase wrap occurs when the fringes move more than one cycle (π), further comprising correcting for the phase wraps using the equation:

Adjusted Value=−π−(π−signal)−n(2π) (3).

In some aspects, the sample from the PCR comprises oligonucleotides and dNTPs that are label-free.

In some aspects, the substrate and channel together comprise a capillary tube. In some aspects, the substrate and channel together comprise a microfluidic device. In some aspects, the microfluidic device comprises a polymeric substrate and an etched channel formed in the substrate for reception of a fluid sample, the channel having a cross sectional shape. In some aspects, the polymeric substrate comprises one or more polymers selected from polycarbonate, polydimethylsiloxane, fluorosilicone, polytetrafluoroethylene, poly(methyl methacrylate), polyhexamethyldisilazane, polypropylene, starch-based polymers, epoxy, and acrylics. In some aspects, microfluidic device comprises a silica substrate and a channel formed in the substrate for reception of a fluid sample, the channel having a cross sectional shape. In some aspects, the channel is formed by etching, by molding, by micromachining, or by photolithography. In some aspects, the cross sectional shape is substantially rectangular, substantially circular, or generally semi-circular. In some aspects, the coherent light beam is produced by a laser.

Detection is enabled by the unique ability for BSI to quantify molecular interactions at very high sensitivity, label-free and in free-solution (Science, September 2007). So since BSI can be used to detect molecular interactions between DNA, by directly illuminating a ‘channel’ in a chip, the presence or absence of a particular (allele) sequence can be determined without the need to label the probe DNA. These individual outputs can then be used to determine the presence or absence of a particular sequence or to map the origin of the starting sample (blood, tissue, etc.) through sequence homology comparisons.

Without wishing to be bound by theory, back-scattering interferometry can measure one or more molecular interactions and/or activities occurring during hybridization. For example, the refractive index (RI) can be affected by the hydrogen bonds formed when the nucleotide hybridizes to the base pair on the complementary strand.

### Oligonucleotide Arrays

An array is an orderly arrangement of samples, providing a medium for matching known and unknown biological molecules, such as DNA samples based on base-pairing rules, and optionally, automating the process of identifying the unknowns. An array of the current invention, which comprises multiplexing of detection regions, may comprise regions within a single channel or across multiple channels. The detection site is the region of the channel that corresponds to the region of the fringe pattern that is being interrogated. A BSI device may function as an array wherein the channel or channels of a BSI device comprises one or more detection sites and may comprise immobilized biological molecular species such as a nucleic acid, a protein, enzyme, receptors, antibody. The arrangement of the detection sites to produce an array of such sites, is within the skill of those knowledgeable in the art, and known array configurations can be used by a BSI device. For example, design of detection regions may mimic known formats such as a biochip, DNA chip, DNA microarray, GeneChip® (Affymetrix, Inc which refers to its high density, oligonucleotide-based DNA arrays), and gene array. Examples of array uses include SNP arrays, and may be used for detecting genetic variation and the source of susceptibility to genetically caused diseases. Generally termed genotyping applications, BSI arrays or multiplex design devices may be used for forensic applications, rapidly discovering or measuring genetic predisposition to disease, identifying DNA-based drug candidates, to profile somatic mutations in cancer, loss of heterozygosity events, amplifications and deletions of regions of DNA, to evaluate germline mutations in individuals, or somatic mutations in cancers, as genome tilting arrays with overlapping oligonucleotides designed to blanket an entire genomic region of interest.

Multiplex or array BSI devices and methods are described in PCT/US2011/039982 and U.S. patent application Ser. No. 13/157,803, each of which is herein incorporated in its entirety. In an aspect, the inventive interferometric detection system and methods are capable of measuring multiple signals, for example, along a length of a capillary channel, simultaneously or substantially simultaneously. In an aspect and while not wishing to be bound by theory, the refractive index changes that can be measured by the multiplexed interferometric detection systems and methods of the present disclosure can arise from molecular dipole alterations associated with conformational changes of sample-ligand interaction as well as density fluctuations due to changes in waters of hydration. The detection system has numerous applications, including the observation and quantification of molecular interactions, molecular concentrations, bioassays, universal/RI detection for CE (capillary electrophoresis), CEC (capillary electrochromatography) and FIA (flow injection analysis), physiometry, cell sorting/detection by scatter, ultra micro calorimetry, flow rate sensing, and temperature sensing. One of the advantages of the systems and methods of the a multiplex or assay design BSI is that a sample measurement and reference measurement can be acquired simultaneously or substantially simultaneously from the same channel. Measurement in multiplex BSI device or a single channel of discrete regions having control and unknown reactions allows for similar conditions, in timing and in space, for both the control and the unknown reactions, which leads to more reliable results. As both measurements occur in the same channel and, in an aspect, in immediately adjacent portions of the channel, the thermal properties and other properties attributable to each measurement will be uniform, resulting in higher signal to noise levels.

In an aspect, a channel can be divided into multiple discrete zones along the length of the channel. Alternatively, multiple channels may be used, and though the description herein is directed to one channel, the present invention comprises assays or multiplex design BSI to include multiple channels. In an aspect, a channel may comprise at least two discrete zones. In an aspect, a channel can comprise 2, 3, 4, 5, 6, 7, 8, 9, 10, or more zones. Any individual zone can have dimensions, such as, for example, length, the same as or different from any other zones along the same channel. In an aspect, at least two zones have the same length. In an aspect, all of the zones along a channel have the same or substantially the same length. In an aspect, each zone can have a length along the channel of from about 1 to about 1,000 micrometers, for example, about 1, 2, 3, 5, 8, 10, 20, 40, 80, 100, 200, 400, 800, or 1,000 micrometers. In an aspect, each zone can have a length of less than about 1 micrometer or greater than about 1,000 micrometer, and the present disclosure is not intended to be limited to any particular zone dimension. Further, any individual zone can be in contact with or separated from an adjacent zone. In an aspect, at least one zone is in contact with an adjacent zone. In an aspect, each of the zones along a channel are in contact such that there are no breaks between individual zones. In an aspect, at least one zone is separated from an adjacent zone by a portion of the capillary not in a zone. In an aspect, each of the zones along a channel are separated from each other such that no zones are in direct contact with another.

In an aspect, at least one zone can be used as a reference and/or experimental control. In an aspect, each measurement zone can be positioned adjacent to a reference zone, such that the channel comprises alternating measurement and reference zones. It should be noted that the zones along a channel do not need to be specifically marked or delineated, only that the system be capable of addressing and detecting scattered light from each zone.

In an aspect, any one or more zones in a channel can be separated from any other zones by a junction, such as, for example, a union, coupling, tee, injection port, mixing port, or a combination thereof. For example, one or more zones in the flow path of a sample can be positioned upstream of an injection port where, for example, an analyte can be introduced. In an aspect, one or more zones can also be positioned downstream of the injection port.

In an aspect, a channel can be divided into two, three, or more regions, wherein each region is separated from other regions by a separator. In an aspect, a separator can prevent a fluid in one region of a channel from contacting and/or mixing with a fluid from another region of the channel. In an aspect, any combination of regions or all of the regions can be positioned such that they will be impinged with at least a portion of the light beam. In an aspect, multiple regions of a single channel can be used to conduct multiple analyses of the same of different type in a single instrumental setup. In an aspect, a channel has two regions, wherein a separator is positioned in the channel between the two regions, and wherein each of the regions are at least partially in an area of the channel where the light beam is incident.

In an aspect, if multiple regions are present, each region can have an input and an output port. In an aspect, the input and/or output ports can be configured so as not to interfere with the generation of scattered light, such as, for example, backscattered light, and the resulting measurements. It should be noted that other geometric designs and configurations can be utilized. Thus, in an aspect, a single channel can allow for analysis of multiple samples simultaneously in the same physical environment.

In an aspect, a separator, if present, comprises a material that does not adversely affect detection in each of the separated regions, such as, for example, by crating spurious light reflections and refractions. In an aspect, a separator is optically transparent. In an aspect, a separator does not reflect light from the light source. In an aspect, a separator can have a flat black, non-reflective surface. In an aspect, the separator can have the same or substantially the same index of refraction as the channel. In an aspect, a separator can be thin, such as, for example, less than about 2 μm, less than about 1 μm, less than about 0.75 μM.

### Molecular Interactions and Biosensor Applications

Molecular interaction analysis is an active area of biomedical research as scientists look for understanding of which molecules bind to other molecules. This information can be critical on any number of levels, especially as it pertains to an understanding of the mechanism of action of pharmaceutical small molecules or biological macromolecules. The study of interactions can also elucidate possible mechanisms of toxicity and can help identify how best to modify molecules to become more effective therapeutics. A thorough understanding of which molecules bind which molecules can also lead to a more comprehensive understanding of the molecular pathways involved in gene function which can help identify new points of intervention in disease states such as cancer or diabetes, or new points of intervention in the pathways that contribute to aging. Molecular interactions can also provide a rapid diagnostic tool for the presence or absence of molecules that are correlated with disease or with the presence of pathogens in the environment.

Biosensors have been defined as any type of device that contains a bioreceptor and a transducer. The bioreceptor can be a biological molecular species such as a nucleic acid, a protein, enzyme, receptors, antibody or even a living biological system such as cells or whole organisms that would bind the target species. The transducer would then convert this binding event into a measurement that could be recorded or displayed. Several types of transducers have been developed, including optical measurements, including fluorescence, luminescence, absorption, phosphorescence, Raman, SERS, surface Plasmon resonance, and back-scattering interferometry, electrochemical, and mass-sensitive (including surface acoustic wave and microbalance). Methods and devices of the present invention comprising BSI function as biosensors, whether in a single channel, a single channel with multiple detection regions, or multiple channel formats.

In conventional nucleic acid biosensors, the specific sequence of bases that define a segment of DNA can be used as a probe to bind other DNA sequences, and these DNA sequences can be labeled with radioactive or other labels. In an aspect, the invention relates to a DNA biosensor because BSI methods and devices, in the absence of a labeled secondary DNA probe, can detect the primary nucleic acid molecules binding to the target nucleic acid molecules, from a change in the refractive index due to the binding event.

Accordingly, in an aspect, the invention comprises a method for free-solution determination of molecular interactions or a method wherein at least a first nucleic acid or other biological molecular species is immobilized within the channel, comprising the steps of providing a substrate having a channel formed therein for reception of a fluid sample to be analyzed, wherein the channel may comprise immobilized nucleic acid molecules or other biological molecular species; optionally, introducing a first sample comprising at least a first non-immobilized nucleic acid polymer or other biological molecular species to be analyzed into the channel; introducing a sample comprising at least a non-immobilized nucleic acid polymer or binding partner to the biological molecular species, to be analyzed into the channel; allowing the first nucleic acid polymer or biological molecular species to interact with the second nucleic acid polymer or binding partner of the biological molecular species to form one or more interaction products; directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; detecting positional shifts in the light bands; and determining the formation of the one or more interaction products of at least the first nucleic acid polymer with at least the second nucleic acid polymer or of at least the first biological molecular species with at least the second biological molecular species, from the positional shifts of the light bands in the interference patterns, wherein the method is employed to detect a DNA sequence of interest in the absence of a labeled secondary DNA probe, or the detection of protein-DNA or RNA interactions, or protein-protein binding, or enzyme-substrate binding, or receptor-ligand binding, or antibody-epitope binding, or to measure one or more characteristic properties and/or chemical events of unlabelled (i.e., substantially label-free) or labeled nucleic acid polymers, or binding of any two biological binding partners. A binding partner pair is the pair of biological molecular species that are known to interact with each other, including but not limited to, a receptor and its ligand(s), complementary nucleic acid molecules, antibody and its epitope(s), an enzyme and its cofactors or substrate.

In an aspect, the invention comprises a method for real-time, free-solution determination of molecular interactions or a method wherein at least one binding partner is immobilized with the channel, comprising the step of detecting the formation of one or more interaction products of at least two biological molecular, species, wherein at least one of the biological molecular species is present during the determination at a concentration of less than about 5.0×10⁻⁵M, wherein the method is employed to detect a DNA sequence of interest in the absence of a labeled secondary DNA probe, or the detection of protein-DNA or RNA interactions, or protein-protein binding, or enzyme-substrate binding, or receptor-ligand binding, or antibody-epitope binding, or to measure one or more characteristic properties and/or chemical events of unlabelled (i.e., substantially label-free) or labeled nucleic acid polymers, or binding of any two biological binding partners.

In an aspect, the invention comprises a method for real-time, free-solution determination of molecular interactions or a method wherein at least one binding partner is immobilized with the channel, comprising the step of detecting the formation of one or more interaction products of at least two biological molecular species, wherein at least one of the biological molecular species is present during the determination in a solution with a volume in the detection zone of less than about 500 nL, wherein the method is employed to detect a DNA sequence of interest in the absence of a labeled secondary DNA probe, or the detection of protein-DNA or RNA interactions, or protein-protein binding, or enzyme-substrate binding, or receptor-ligand binding, or antibody-epitope binding, or to measure one or more characteristic properties and/or chemical events of unlabelled (i.e., substantially label-free) or labeled nucleic acid polymers, or binding of any two biological binding partners.

In an aspect, the invention comprises a method for free-solution determination of molecular interactions or a method wherein at least one biological molecular species is immobilized within the channel, comprising the steps of providing a substrate having a channel formed therein for reception of a fluid sample to be analyzed, wherein the channel may comprise immobilized nucleic acid molecules or other biological molecular species; optionally, introducing a first sample comprising at least a first non-immobilized nucleic acid polymer or biological molecular species to be analyzed into the channel; establishing a baseline interferometric response by directing a coherent light beam onto the substrate such that the light beam is incident on the channel to generate backscattered light through reflective and refractive interaction of the light beam with a substrate/channel interface and the sample or the immobilized biological molecular species, the backscattered light comprising interference fringe patterns including a plurality of spaced light bands whose positions shift in response to changes in the refractive index of the first sample; introducing a sample comprising a mixture of the first non-immobilized nucleic acid polymer or biological molecular species and a second non-immobilized nucleic acid polymer or binding partner to the biological molecular species to be analyzed, wherein the first nucleic acid polymer or biological molecular species interacts with the second nucleic acid polymer or binding partner of the biological molecular species to form one or more interaction products, into the channel; directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the second sample; detecting positional shifts in the light bands relative to the baseline; and determining the formation of the one or more interaction products of the first nucleic acid polymer with the second nucleic acid polymer from the positional shifts of the light bands in the interference patterns, wherein the method is employed to detect a DNA sequence of interest in the absence of a labeled secondary DNA probe, or the detection of protein-DNA or RNA interactions, or protein-protein binding, or enzyme-substrate binding, or receptor-ligand binding, or antibody-epitope binding, or to measure one or more characteristic properties and/or chemical events of unlabelled (i.e., substantially label-free) or labeled nucleic acid polymers, or binding of any two biological binding partners.

In an aspect, the invention relates to an interferometric detection system comprising a substrate; a channel formed in the substrate for reception of a fluid sample to be analyzed or a method wherein at least one binding partner is immobilized with the channel; means for introducing a first sample comprising at least a first nucleic acid polymer or other biological molecular species; means for introducing a second sample comprising at least a second nucleic acid polymer or binding partner of the biological molecular species; optionally, means for mixing the first sample and the second sample; a coherent light source for generating a coherent light beam, the light source being positioned to direct the light beam onto the substrate such that the light beam is incident on the channel to thereby generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; a photodetector for receiving the backscattered light and generating one or more intensity signals that vary as a function of positional shifts of the light bands; and a signal analyzer for receiving the intensity signals, and determining therefrom, a characteristic property of the fluid sample in the channel, wherein the method and device is a biosensor.

In an aspect, BSI can measure label-free molecular interactions. One example of a label-free measurement in life science applications can be when the BSI device is used to interrogate the binding of two biological molecular species, such as a DNA binding protein and the fragment of DNA that contains the sequence that the protein binds by examining a change in the interference pattern produced from the reflection and refraction of the solution upon mixing the two biological macromolecules. Known are methods that require DNA oligonucleotides to be immobilized prior to measuring the binding of a single-stranded DNA binding protein which was visualized using surface plasmon resonance (1999 JACS Brockman et al., 121:8044-51). In contrast, a BSI method does not require that the protein being examined be labeled or be bound to a solid support, though such steps may be used in a BSI device or method, as the measurement could be made in free solution or using immobilized biological molecular species in a BSI.

For the detection of biomolecular interactions, the following types of detectors can be replaced by BSI methods and/or devices or can be able to be used in combination with BSI methods and/or devices, including optical techniques including Surface enhanced Raman spectroscopy, and Surface Plasmon Resonance (SPR). SPR is an optical phenomenon used for measuring molecular interactions but requires that one molecular species be immobilized. The SPR signal arises in thin metal films and the signal depends on the refractive index of solutions in contact with the metal surface. A challenging aspect of using SPR is direct immobilization of one of the molecular species without disrupting its binding activity. In contrast to SPR, BSI methods and/or devices can be used to measure the binding of macromolecules without either macromolecule being fixed to a surface. For example, using SPR, it was recently shown that soluble monomeric beta-amyloid peptides can bind anti-beta-amyloid monoclonal antibodies (J Phys Chem B 2007; 111: 1238-43). In contrast, BSI methods and/or devices can also be used to measure soluble monomeric beta-amyloid peptides binding an anti-beta-amyloid monoclonal antibodies in free solution.

A type of detector that can be replaced by BSI methods and/or devices or used in combination with BSI methods and/or devices is one that utilizes grating based approaches such as optical waveguide lightmode spectroscopy (OWLS). OWLS measures the surface immobilization of biomolecules in an aqueous solution. The technique is based on the incoupling of a laser into a waveguide by an optical grating. The incoupling only occurs at two defined angles that are sensitive to a change in the refractive index above the surface in the evanescent field. The OWLS method uses the change in the refractive index to measure the adsorbed mass. A challenging aspect of using OWLS is direct immobilization of one of the molecular species. In contrast to OWLS, BSI methods and/or devices can be used to measure the binding of macromolecules without either macromolecule being fixed to a surface. For example, using OWLS, the interaction between mycotoxins and anti-mycotoxin monoclonal antibodies was measured (Biosens Bioelectron 2007 22:797-802). In contrast, BSI methods and/or devices can also be used to measure the binding of soluble mycotoxins binding anti-mycotoxin monoclonal antibodies in free solution, or by having one of the biological molecular species immobilized to a substrate.

A type of detector that can be replaced by BSI methods and/or devices or used in combination with BSI methods and/or devices is one that utilizes mass-sensitive measurements such as surface acoustic wave (SAW). In SAW, small mass changes can be measured that result from molecules binding the receptor molecules coupled to the active sensor surface. Small mass changes at the sensor surface affects the propagation velocity of acoustic shear waves traveling through a guiding layer at the sensor surface. A challenging aspect of using SAW is direct immobilization of one of the molecular species. In contrast to SAW, BSI methods and/or devices can be used to measure the binding of macromolecules without either macromolecule being fixed to a surface. For example, using SAW, the interaction between bovine immunoglobulin G and Protein A was recently measured (International Conference on Solid State Sensors and Actuators June 16-19 1997 1:187-190). In contrast, BSI methods and/or devices can also be used to measure the binding of bovine immunoglobulin G and Protein A in free solution, or by having one of the biological molecular species immobilized to a substrate.

A type of detector that can be replaced or used in combination with BSI methods and/or devices is one that utilizes mass-sensitive measurements utilizing a piezoelectric crystal. For example, small mass changes can be measured that result from molecules binding the receptor molecules coupled to the active sensor surface due to a change in the oscillation frequency of a piezoelectric crystal. Piezoelectric crystals oscillate as a function of both the electrical frequency applied to the crystal and the crystal's mass. Small mass changes can therefore be measured electrically. In contrast to a microbalance, BSI methods and/or devices can be used to measure the binding of macromolecules without either macromolecule being fixed to a surface. For example, using a piezoelectric crystal, the interaction between Staphylococcal Enterotoxin B (SEB) and anti-SEB polyclonal antibodies was measured (Biosens Bioelectron 1997 12:661-7). In contrast, BSI methods and/or devices can also be used to measure the binding of Staphylococcal Enterotoxin B and anti-SEB polyclonal antibodies in free solution, or by having one of the biological molecular species immobilized to a substrate.

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which may need to be independently confirmed.

### DEFINITIONS

As used in the specification and the appended claims, the singular forms “a,” “an” and “the” include plural referents unless the context clearly dictates otherwise. Thus, for example, reference to “a substrate,” “a polymer,” or “a sample” includes two or more such substrates, polymers, or samples, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where the event or circumstance occurs and instances where it does not.

As used herein, the term “polymer” refers to a high molecular weight organic compound, natural or synthetic, whose structure can be represented by a repeated small unit, the monomer, for example nucleic acid polymers, made from nucleotides.

As used herein, the term “copolymer” refers to a polymer formed from two or more different repeating units (monomer residues). By way of example and without limitation, a copolymer can be an alternating copolymer, a random copolymer, a block copolymer, or a graft copolymer.

As used herein, the term “bioassay” refers to a procedure for determining the concentration, purity, and/or biological activity of a substance.

As used herein, the term “chemical event” refers to a change in a physical or chemical property of an nucleic acid polymer in a sample that can be detected by the disclosed systems and methods. For example, a change in refractive index (RI), solute concentration and/or temperature can be a chemical event. As an example, a biochemical binding or association (e.g., DNA hybridization) between two chemical or biological species can be a chemical event. That is, a chemical event can be the formation of one or more interaction products of the interaction of a first nucleic acid polymer with a second nucleic acid polymer, referred to herein as a hybridization product. As an example, a disassociation of a complex or molecule can also be detected as an RI change. As an example, a change in temperature, concentration, and association/dissociation can be observed as a function of time. As an example, bioassays can be performed and can be used to observe a chemical event.

As used herein, the terms “equilibrium constant” and “K_{c}” and “K_{eq}” refer to the ratio of concentrations when equilibrium is reached in a reversible reaction. For example, for a general reaction given by the equation:

aA+bB⇄cC+dD,

the equilibrium constant can be expressed by:

$${K}_{c} = \frac{{{[ C ]}^{c} [ D ]}^{d}}{{{[ A ]}^{a} [ B ]}^{b}} .$$

An equilibrium constant can be temperature- and pressure-dependent but has the same value, irrespective of the amounts of A, B, C, and D. A specific type of equilibrium constant that measures the propensity of a larger object to separate (dissociate) reversibly into smaller components is a “dissociation constant” or “K_{D}.” A dissociation constant is the inverse of an “affinity constant.”

As used herein, the term “dissociation rate” is a concentration dependent quantity and involves the “dissociation rate constant” or “K_{D}.” The dissociation rate constant relates the rate at which molecules dissociate to the concentration of the molecules. A dissociation can be described as AB→A+B, and the rate of dissociation (dissociation rate) is equal to k_{D}[AB]. In general, the larger the value of k_{D}, the faster the inherent rate of dissociation.

As used herein, the term “association rate” is a concentration dependent quantity and involves the “association rate constant” or “k_{A}.” The association rate constant relates the rate at which molecules associate to the concentration of the molecules. An association can be described as A+B→AB, and the rate of association (association rate) is equal to k_{A}[A][B]. In general, the larger the value of k_{A}, the faster the inherent rate of association.

As used herein, the term “free-solution” refers to a lack of surface immobilization. The term is not meant to exclude the possibility that one or more molecules or atoms of nucleic acid polymer may associate with a surface. Rather, the term can describe the detection of an nucleic acid polymer without the requirement for surface immobilization during analysis.

As used herein, the term “label-free” describes a detection method wherein the detectability of an nucleic acid polymer is not dependent upon the presence or absence of a detectable label. For example, “label-free” can refer to the lack of a detectable label. It is understood that the ability of a label to be detected can be dependent upon the detection method. That is, an nucleic acid polymer having a moiety capable of serving as a detectable label for a first detection method can be considered “label-free” when a second detection method (wherein the label is not detectable) is employed. In an aspect, the nucleic acid polymers employed in the disclosed systems and methods can lack detectable labels.

As used herein, the term “detectable label” refers to any moiety that can be selectively detected in a screening assay. Examples include without limitation, radiolabels (e.g., ³H, ¹⁴C, ³⁵S, ¹²⁵I, ¹³¹I), affinity tags (e.g. biotin/avidin or streptavidin), binding sites for antibodies, metal binding domains, epitope tags, FLASH binding domains (see U.S. Pat. Nos. 6,451,569; 6,054,271; 6,008,378 and 5,932,474), glutathione or maltose binding domains, photometric absorbing moieties, fluorescent or luminescent moieties (e.g. fluorescein and derivatives, GFP, rhodamine and derivatives, lanthanides etc.), double strand nucleic acid dyes, and enzymatic moieties (e.g. horseradish peroxidase, β-galactosidase, β-lactamase, luciferase, alkaline phosphatase). Detectable labels can be formed in situ, for example, through use of an unlabeled primary antibody which can be detected by a secondary antibody having an attached detectable label. Further examples include imaging agents such as radioconjugate, cytotoxin, cytokine, Gadolinium-DTPA, a quantum dot, iron oxide, and manganese oxide.

Disclosed are the components to be used to prepare the compositions of the invention as well as the compositions themselves to be used within the methods disclosed herein. These and other materials are disclosed herein, and it is understood that when combinations, subsets, interactions, groups, etc., of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### An Example of BSI Device Fabrication

A fluidic network was designed using commercially available software (CleWin 2.7). A soda lime/chrome lithographic mask (chrome thickness approximately 100 nm) was then prepared (Delta Mask, The Netherlands) using this fluidic network design. Master molds were subsequently created from the lithographic mask using conventional optical and soft lithographic techniques.

Three inch silicon wafers (P<100>) were cleaned by sonication in acetone followed by treatment with piranha solution. The sonicated and treated wafers were then rinsed with deionized water and placed on a hot plate at 95° C. for 5 minutes just prior to deposition of a photoresist. A negative photoresist (SU-8 2050, available from Microchem, Newton, Mass., USA) was then evenly deposited on the surface of the Si wafer using a bench-top single wafer spinner (Laurell WS-400). A few milliliters of the negative photoresist were poured onto the center of the wafer and spinning commenced for 10 seconds at 500 rpm to spread the photoresist. The speed of the wafer was then increased to 3000 rpm for 40 seconds to form a homogeneous coating. The wafer was then removed from the spin coater and placed on a hot plate for a soft bake (3 min at 65° C. [symbol] 9 min at 95° C.). The wafer was subsequently allowed to cool to room temperature. UV exposure through the photolithographic mask for ˜15 seconds was accomplished using a Laurell WS-400 Bench-top single wafer spinner contact mask aligner. Following irradiation, a post exposure bake (PEB) was performed (1 min at 65° C. 7 min at 95° C.). The wafer was again cooled to room temperature.

Unexposed areas of photoresist were then removed using an organic developer (SU-8, available from Microchem). Isopropyl alcohol (IPA) was used to ensure the wafer was completely developed. IPA will form a milky white substance on the wafer if any unexposed photoresist remains. The master mold was then rinsed and hard baked (˜5 hours at 2200° C.) to ensure device stability. An Alphastep 200 stylus surface profiler (Tencor Instruments) was used to accurately measure the height of the standing relief structures.

Cast molding was performed using a silicon elastomer, polydimethylsiloxane (PDMS), purchased as Sylgard 184 (Dow Corning, Midland, Mich.). Prior to casting, the PDMS was mixed in a 10:1 ratio (base:curing agent) and degassed.

PDMS was cast over the master that had been placed into a 100×15 mm Falcon Petri dish (Becton Dickinson, Franklin Lakes, N.J.) such that the height of the PDMS was ˜2 mm. The Petri dish was placed into a desiccator, and a vacuum was applied for further degassing. Once no air bubbles were visibly present, the Petri dish was removed from the desiccators and set in a large convection oven for roughly 8 hours at 65° C.

After the curing process was complete, the Petri dish was removed from the oven and allowed to cool briefly. The PDMS microchip device was physically removed from the Si master mold by fine precision scalpel and tweezers. Access ports for sample introduction (2 ports) and applied vacuum/waste removal (1 port) were mechanically punched out by stainless steel capillary tubing. PDMS, with the fluidic network facing up, was then plasma oxidized for ˜10 sec along with a 3″×1″×1 mm microscope glass slide (Fisher Scientific) cleaned in the same fashion as the bare Si wafer. Following oxidation, the PDMS was sealed to the microscope slide so that the fluidic network was in contact with the glass. Water was kept in the channels molded in the PDMS until experiments were run to help maintain the hydrophilic surface created by plasma oxidation.

### DNA Hybridization

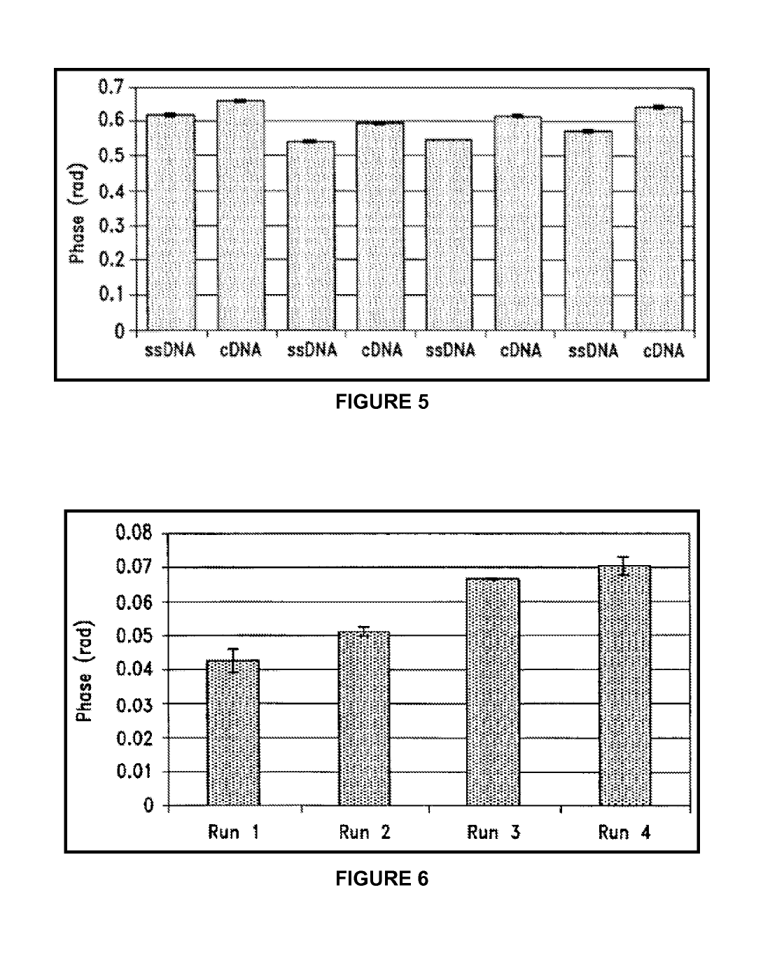

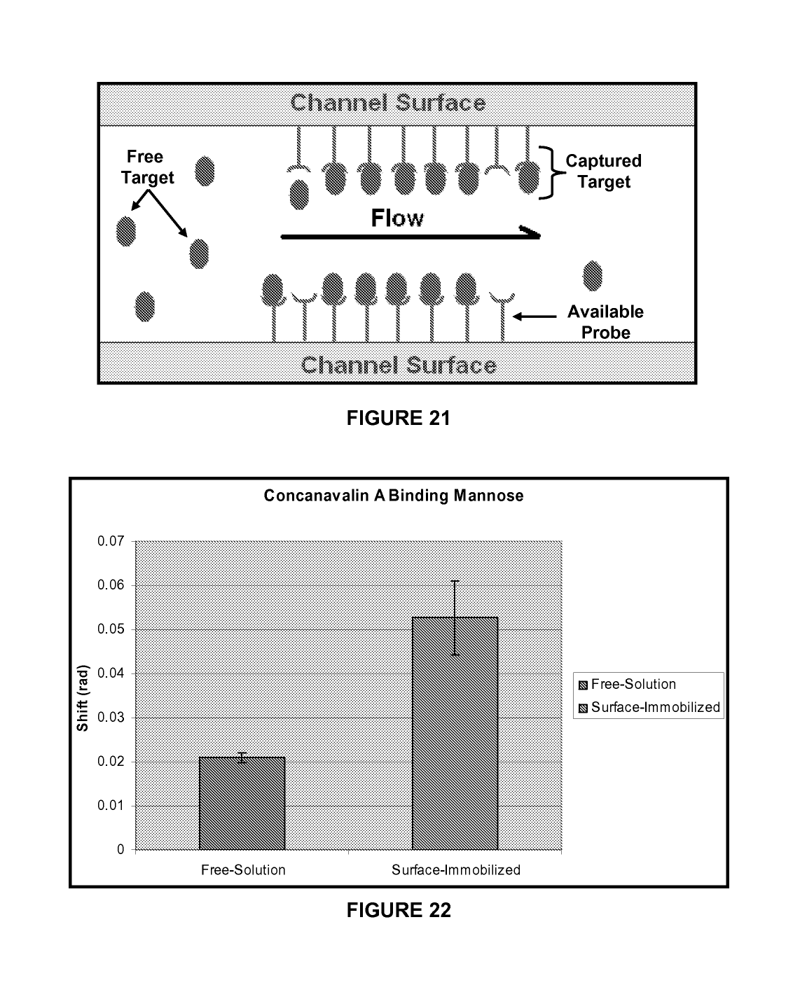

Hybridization of single stranded DNA (ssDNA) to its complimentary strand (cDNA) were performed in 50 μm×50 μm rectangular microfluidic channels molded in PDMS in the probe volume of 2.5×10⁻¹⁰ L. The mouse Actin ssDNA surface immobilization was performed in three steps: first a photoactive form of biotin was deposited onto channel walls and activated with UV light; then avidin was introduced into the channel and allowed to react with immobilized photobiotin; next injected biotinalated ssDNA was allowed to react with the immobilized avidin. A 2048-element array in combination with Fourier analysis was used to quantify the positional change of the fringe pattern. The change in absolute signal due to hybridization and denaturization is shown in FIG. 5. Using the single-channel configuration of OCIBD and when reaction kinetics are not desired, the signal is recorded in two stages: a) when only ssDNA present on the channel surface and the fluid within the channel is the PBS buffer; and b) after the introduced cDNA strand has fully reacted with the immobilized ssDNA strand and after the PBS buffer has been reintroduced into the channel. This approach allows for the elimination of erroneous results due to bulk RI changes from the target species solution. From the signal magnitude of a determination, the analytical utility is demonstrated with a simple calculation. Using the parameters: Avidin dimensions of 5.6×5 nm, a probe volume of 2.5×10⁻¹⁰ L, Avogadro's number, and based on the worst-case scenario assumption that 100% of the surface is covered with avidin and 100% of it is reacted with ssDNA 1.2×10⁻¹⁶ mol (12 fmol) of bound DNA can be reliably detected. As shown in FIG. 21 this determination gives a result with a relatively large signal to noise (S/N) ratio. Further interrogation of the data suggests the S/N=13, so the 36 detection limits would be 3 fmol of target DNA reacting with its counter part. These results represent an approximately two-decade improvement over SPR.

### Example 2

### Materials and Methods

BSI was performed similarly to previous reports (Markov, D., et al. 2004; Latham, J. C., et al. 2006; Bornhop, D. J., et al. 2007) to measure the amount of DNA replicated during PCR, with several changes, related to the way the data were processed due to substantial shifts in the fringe pattern which are discussed below. In short, a vertically mounted chip was illuminated by a He:Ne laser filtered by a neutral density filter before impinging the channel. The chip was fabricated (Micronit Microfluidics BV, Netherlands) out of borosilicate with channels 210 μm wide by 100 μm deep in a semi-circular shape (the probe volume was just 13.4 nL). A 50 μl glass syringe with a flat tip metal needle introduced the sample into the chip via fused silica capillary and appropriate fittings. The fringe pattern generated was redirected with a mirror onto an Ames camera. A program designed in LABVIEW was used to monitor the frequency of the fringes hitting the camera using a Fast Fourier transform. The procedure for the PCR was as follows:

The final volume of the PCR mixture was 25 μl composed of 1 μl of each primer, 2 μl of template DNA, 0.5 μl of dNTPs, 5 μl of 5× Green GoTaq buffer, 0.125 μl of Taq polymerase, and 15.38 μl of sterile DNA free water.

The samples were then run on the thermocycler with the following parameters: (1) 94 degrees C. for 2 minutes, (2) 94 degrees C. for 1 minute, (3) 54 degrees C. for 1 minute, (4) 72 degrees C. for 5 minutes, (5) 72 degrees C. for 10 minutes, and (6) hold at 4 degrees C. Steps (2) to (4) are the “cycles” of the PCR. Generally, the extension time (4) can be shorter than 5 minutes, but this time was chosen given the primers being used. The primers were designed to amplify a region in the DNA which is roughly 3000 base pairs.

In a first set of experiments, an aliquot was taken out after cycle numbers 2, 4, 6, 8, 10, and 30. These samples were then run on a gel to compare the results with those measured by BSI of the same samples. The gels were a 0.8% agarose gel with ethidium bromide. The ethidium bromide intercalates into DNA to make it visible when illuminated with UV light. The gels were run in tris-acetate-EDTA (TAE) buffer at 100 volts for one hour. Pictures of the gels were taken using UV light.

In a second set of experiments three sets of controls were also run to ensure the PCR protocol was performing properly and actual DNA replication was being observed by BSI. The first control was a full PCR mixture with extra buffer instead of DNA. The second control was a full PCR mixture with non-compatible primers which are specific for DNA sequences that are too far apart to allow successful amplification. The third control contained starting DNA which was diluted by a factor of ten and the same incompatible primers. The three sets of controls were analyzed using gels and BSI.

PCR was then performed with two DNA samples with the correct primers. The first sample contained roughly 1,000,000 copies of DNA (S. aureus with 2000 bases in a volume of 25 μl of PCR mixture giving a concentration of around 70 femtomolar (fM). The second sample of DNA used was diluted by a factor of ten. Both sets were cycled and analyzed using gels and BSI.

To prepare the channel for the beginning of the run, 5× Green GoTaq buffer, at a concentration which is the same as used in the PCR procedure, was injected repeatedly into the channel until the standard deviation of the injections is roughly 0.02 phase shift. Once this stability is achieved, the samples were introduced into the channel sequentially starting with the sample from cycle 0. Once the run was completed, the process was repeated for triplicate measurements.

Results

The results of the preliminary experiments indicated that BSI can be used as a label-free, free-solution, ultra-small volume, high sensitivity PCR detection methodology.

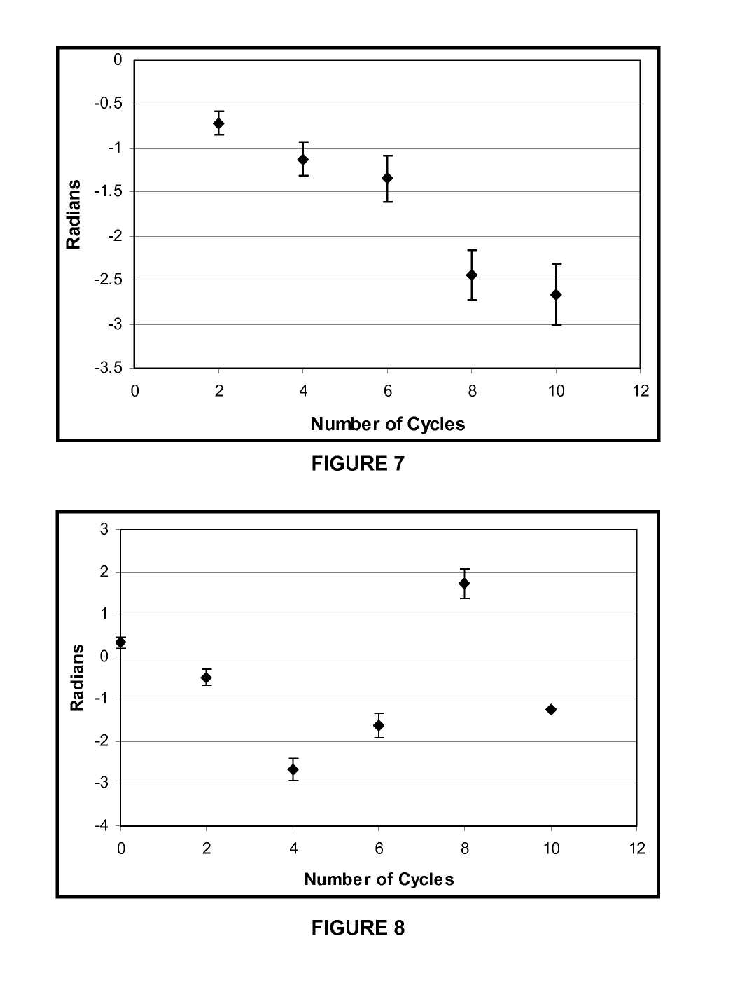

A gel from the first set of experiments showed amplification was successful, but due to sensitivity limitations, a band was not be observed before cycle 25-30. In contrast, label-free, free-solution detection/quantification of PCR is shown in FIG. 7. In these investigations the BSI apparatus was slightly modified using a glass chip with an isotropically etched semicircular channel with an effective diameter of about 100 μm by 210 μm. The increase in probe volume from about 350 pL to about 40 nL leads to an enhanced signal-to-noise ratio, less difficulty in sample introduction and clogging, without a substantial increase in required sample volume. Here the optical alignment was not optimized, so even though there is a significant signal, improvements are possible as shown below. FIG. 7 indicates that there is a quantifiable change in BSI signal as a function of cycle number. The magnitude of change in BSI signal of >2.0 radians corresponds to quite large signal (ΔRI>4.0×10⁻³).

A gel showed the band was very distinct after 30 cycles indicating successful amplification of only the desired DNA fragment. After “tweaking” BSI, obtaining an improvement of nearly 10-fold, a second analysis was performed. The corresponding BSI results from the set of PCR samples from the second experiment are shown in FIG. 8. The “zero” or control data point is not presented in the plot because this set of samples did not contain DNA, primers, and Taq polymerase. While the run-to-run reproducibility was modest giving a bit of a noisy result, probably due to non-specific binding of the DNA to the microfluidic channel, the data showed that even after a few PCR cycles, amplification has occurred. Going from 2 to 4 cycles a shift of about 2.3 radians ({−0.5−(−2.8)}) was detected. For reference, a signal of this magnitude corresponded to a change in RI of 4.5×10-3 RIU.

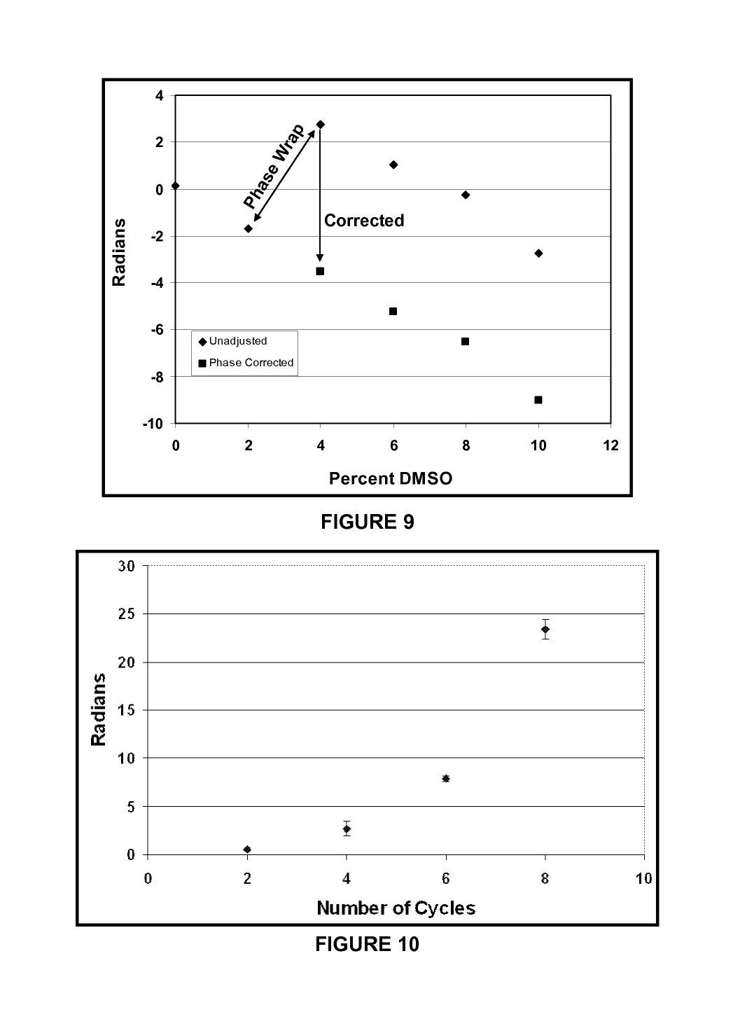

When analyzing Cycle 6 (FIG. 8) it appeared to have less signal than for Cycle 4 based on the radians. This “anomalous” behavior could have been a result of a limitation of the fringe shift detection method that ultimately limited the dynamic operating range of BSI, a tractable problem that can be addressed with a fringe counting approach. In BSI, the fringes shift as the RI changes. The larger the change in signal, the more the fringes will shift (move across the camera used to detect position). An example of signal wrapping is shown in FIG. 9 which demonstrates the BSI-FFT output for a relatively large change in RI (1-10% DMSO standards). Because the phase of the fringes is measured, as they begin to move off the camera or become the adjacent fringe from a large concentration change (large signal), the signal suddenly becomes positive rather than becoming more negative. This “phase wrap” occurs when the fringes move more than one cycle (π) and is depicted or observed in FIG. 9 when going from a DMSO concentration of 2% to 4%. In other words, a phase wrap occurs when one entire fringe moves off (one side) of the camera, and another fringe moves onto the other side of the camera.

For large changes in RI, particularly for macromolecules such as DNA and as expected for exponentially increasing numbers of copies of DNA as in PCR, the fringes shift significantly. In fact, for larger cycles it has been observed visually that fringes sweep across the camera being replaced by adjacent fringes several times, which results in several phase wraps. Under these circumstances (PCR) a “fringe counting” methodology, can be used to quantify the copy number for larger cycle number. Upon close observation of the fringes as the sample is introduced, this fringe sweeping phenomenon was observed. In order to estimate the BSI signal for higher PCR cycle numbers, a phase correction was employed. This phase correction can be used when the signal goes below −π when using an FFT to select a frequency (group of fringes) and then measuring the phase change of that frequency to extract a positional shift of that group of fringes. When the signal goes past −π, it then appears at π. This looping can happen numerous times for very large changes in RI and without watching the fringes shift temporally, it can appear that there was little, no or even negative shift (signal).

To correct for phase wraps, the following equation can be used:

Adjusted Value=−π−(π−signal)−n(2π) (3)

With this equation, the difference between π and the recorded signal is tacked onto −π to give what the true signal should be, and n represents the number of additional times the signal may have wrapped (as is the case for larger PCR cycle numbers). FIG. 9 demonstrates how the response is linearized when the concentration of solute leads to a large change in signal (RI change).

This correction was applied to cycles 6 and 8 of the data set from FIG. 8. Justification for performing a phase wrap correction with the BSI results from the second experiment is based upon several observations. First, the DNA fragment was clearly shown to be amplified during PCR as demonstrated by the gel. Second, the signal observed in going from cycle 2 to 4 was reproducible and quantifiable in the normal operating range of the instrument. Third, BSI response was as expected for an increase in RI (based on DNA and Glycerol calibration curves and in the currently alignment) producing a more negative signal, which should also be the case for an increasing number of DNA fragments. Fourth, the visual confirmation by the user of fringes moving off one side of the camera and others moving onto the camera from the other side (sweeping across the image plane). If you can see the fringes move, there is a very large change in RI being sensed.

FIG. 10 shows the BSI data of the samples from the second experiment with appropriate phase corrections. Assuming that the cycle 2-4 result represented a quantifiable shift (signal), this value corresponded to an increase in the number of DNA strands by 4 fold. Two more cycles would produce an approximate doubling twice or a 4 fold increase of the change from cycle 4 to 6 {i.e., 16 fold increase going from 2 to 6 cycles}. Since the signal amplitude went from about −2.8 radians to −1.8, then the phase wrap was estimated to be slightly less than 2 pi. Here one of the fringes was observed to move about one cycle, becoming the adjacent fringe which would correspond to 2π phase change. Applying the phase wrap adjustment equation gives −π−(π−1.8)−0 (2π)=−8.1 rad. Comparing this to what is ‘theoretically predicted, if 2-4 cycles gave a 2.3 radian change (−0.5−(−2.8)) then two more cycles should give a 4 fold increase in DNA copies or a 4(−2.3)=−9.2 rad signal. For the 8^{th} cycle doubling 2 times more would give a signal of (9.2 (4))=36.8 radians, which is a huge signal. But when introducing this sample into BSI, numerous fringes passing the imaging plane were observed. If the wrap number is proportional to copy number, then if amplification from 4-6 cycles produces about one phase wrap then doubling twice (4 fold amplification) would give 4 wraps. Taking the signal from the graph in FIG. 8 and applying the adjustment equation −π−(π−1.3)−3(2π)=−26.4 rad. Undeniably a large signal, but recall many fringes were observed to shift across the camera. Here under estimate the theoretical value to greater extent, with 4 more cycles predicted to give a (9.2)4=36.8 radian signal.

In summary, the detection of PCR label-free in free-solution was successful, with the signal generally becoming more negative in an exponential manner correlating with the amount of DNA which is growing exponentially during PCR.

A second set of determinations (in triplicate) were performed in a similar manner to those described above, but in this case a number controls were introduced. The controls were; 1) no DNA, 2) DNA with non-complementary primers, and 3) 1:10 dilution of the DNA with the non-complementary primers.

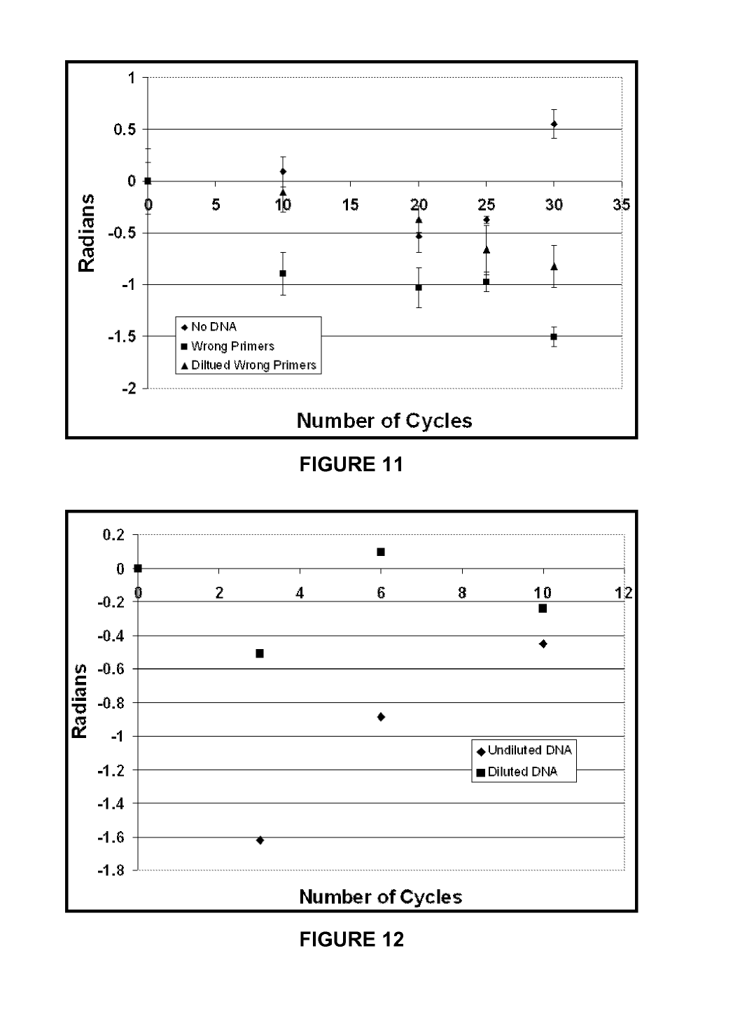

FIG. 11 displays the BSI signals (raw data) for the three controls. In all three controls there should be no DNA amplified, and while a slight signal was seen, it remained mostly constant with cycle number. Amplification was seen in the undiluted and diluted DNA. The data was plotted as a relative change in BSI signal (radians); therefore the signal of the controls at zero cycles start at the same point, e.g. zero is the starting point for each of the measurements. The data were plotted as relative changes in RI. The control with no DNA present gave a signal which moves up and down randomly. This was what is expected and the jumping around in signal could be due to polymerization of primers with nucleotides or themselves. Both of the controls containing incompatible primers showed a gradual, but quite modest increase in signal. This could have been due to some smaller sections of DNA being erroneously amplified. In all cases, close visual observation of the fringes confirmed that there was no “detectable by naked-eye” fringe shift. Even though there appeared to be some slight signal from the sample containing an incompatible primer pair, being able to quantify allowed for correction for its contribution to the signal when smaller numbers or shorter pieces of DNA have to be detected by BSI.

FIG. 12 contains the data from BSI with respect to cycle number for the undiluted and diluted DNA samples. This was the raw data, before any phase wrap corrections have been applied. Shown are cycles 3, 6, and 10 of the samples containing DNA. The plot showed that a large change in signal was detected by BSI upon going from the start (0 cycles) to 3 cycles. As a reference, this shift of 1.6 radians was comparable to that quantified when going from 1 mM to 150 mM glycerol. Note that we were amplifying about 10⁶ copies of a 2000 base pair strand of DNA. With reference to FIG. 12, looping can happen numerous times for very large changes in RI and without watching the fringes shift temporally, it can appear that there was little, no or even negative shift (signal). Here, as with observations that were made in the experiment above, large movement of the fringes were visibly seen at larger cycle number, indicating phase wrapping. Therefore similar phase corrections were performed as described above.

As before, the major assumption being made was the early cycle signal (from 0 to 3) was real and quantifiable by BSI. This assumption was backed up by the observations that; 1) according to the gel the DNA was being amplified, 2) the signal was moving in the correct direction for increasing refractive index and 3) visual observation of sweeping of fringes across the camera sensor. The assumption also correlated well with what the data were showing. The change in signal from 0 to 3 cycles was 1.6 radians. Knowing that PCR will increase the concentration of the DNA segment 8-fold from cycle 3 to 6 (PCR doubles the segment each cycle), the signal should also increase by 8-fold. Multiplying the signal from cycle 3 by 8 gives 12.8 radians, an estimate of what the signal should be for cycle 6. If the recorded signal for cycle 6 (0.88 radians) has wrapped not once but twice (n=1), then the result is 11.7 radians. The difference between the two numbers was likely within the error of the measurement and acceptable because the estimate assumed 100% efficiency for PCR which was unlikely.

When going to 10 cycles, the estimate was found by multiplying 1.62 (actual change in signal for cycles) by 128, giving 207 radians as the signal for 10 cycles. By assuming the number of phase wraps also doubles with each cycle, the corrected signal for cycle 10 was 200 radians. The value of n in this case would be 31 since 2 wraps increased to 32 over 4 cycles. This value agreed very well with the estimate and continued the trend for PCR of growing exponentially.

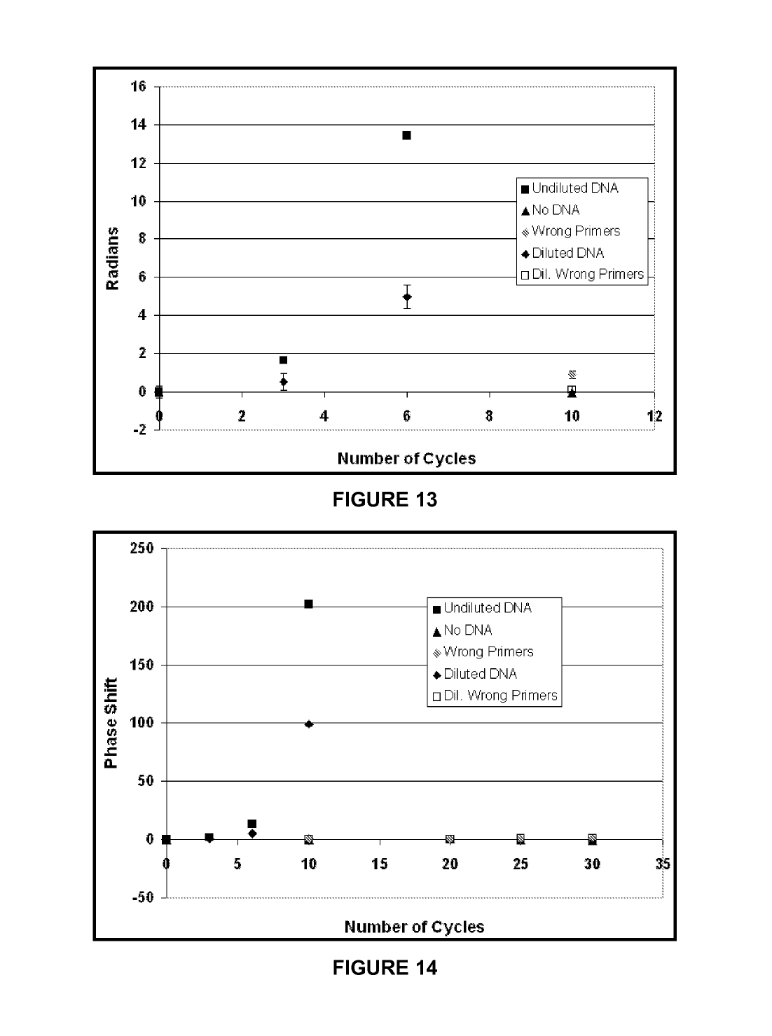

The corrections for the diluted samples followed the same line of reasoning and are shown in FIG. 13. The signal change in going from 0 to 3 cycles was assumed to be real based on observations previously described. The change in signal from 0 to 3 cycles was 0.51 radians. In going from 3 to 6 cycles the number of DNA copies should increase 8-fold. Multiplying the signal from 0 to 3 cycles by 8 gave 4.1 radians as an estimate of what the signal should be. By using the signal for the 6 cycle sample, −0.09, and applying a one phase wrap correction, the result was 4.9 radians. This agreed fairly well with the estimate, and the difference was probably due to high noise in the signal. When applying the same reasoning for cycle 10, 0.51 was multiplied by 128 to give an estimate of 66 radians. Using the data acquired at cycle 10 and assuming the number of phase wraps increased at the same rate as DNA copies (1 to 16 wraps), the result was 99 radians with n=1. The discrepancy was due to the high level of noise in the signal.

The signal for the undiluted samples in this third experiment was much higher than the signal for the samples in the second experiment. There were two possible explanations for these discrepancies. First, the amount of starting DNA could have been different for each sample set. The other reason could be due to BSI performance improvements with the instrument constantly being optimized for the detection of DNA, e.g., the system was better aligned for these types of measurements.

FIG. 14 presents the corrected values for the BSI measurements of PCR up to 10 cycles and all of the controls (out to 30 cycles) showing the relative magnitude in signal for the samples versus the references. In summary this confirmed the ability to perform label-free, free-solution, nanoliter volume PCR with BSI.

### Example 3

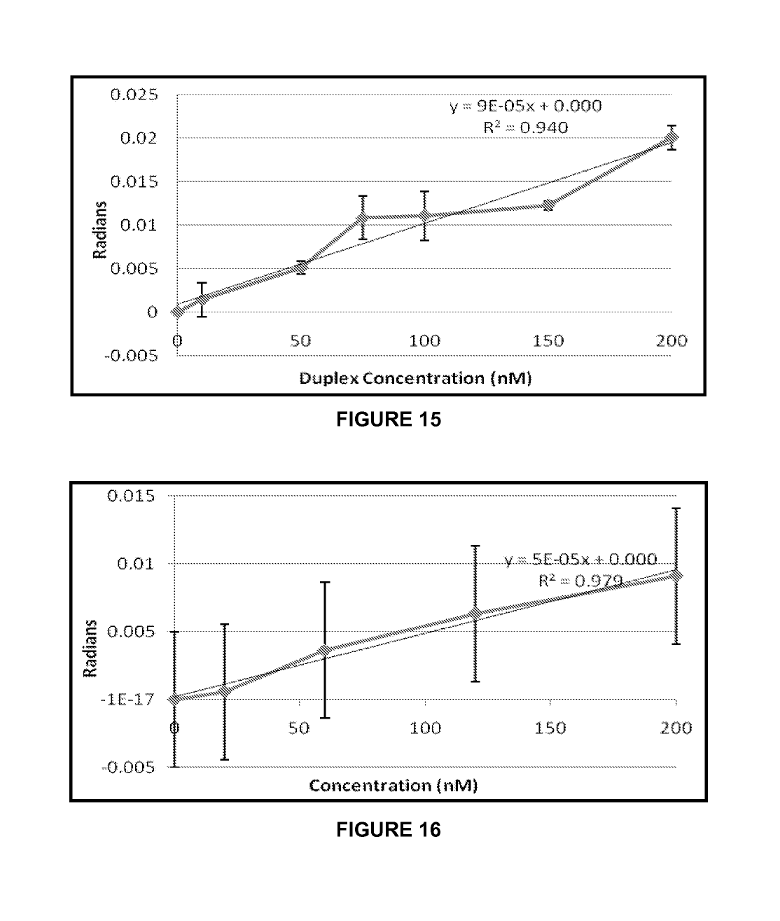

In the present study, BSI was used to monitor the strength of oligonucleotide hybridization directly comparing results obtained from free-solution and surface-immobilized experiments. A 30 oligonucleotide probe strand (Ps) was hybridized to five different 30mers. These included an unlabeled perfectly complementary strand, a complementary 5′-labeled strand tagged with either fluorescein isothiocyanate (FITC) or cyanine-3 (Cy3), and two mismatched strands containing either a terminal CA/AC mismatch or an internal AG/GT mismatch. The steady-state, free-solution BSI experiments were carried out in silanized channels to prevent non-specific oligonucleotide absorption to the surface. Prior to the experiment, 300 nM Ps was mixed with equal volume of serially-diluted target strands ranging from 0-600 nM. See FIG. 15 which shows free-solution BSI unlabeled oligonucleotide duplex calibration. 150 nM was chosen as the constant strand concentration based on the magnitude of the signal shift and comparable concentration to surface-immobilized studies. See FIG. 16 which shows unlabeled oligonucleotide calibration curve which showed a small linear increase in signal due to increasing single stranded concentration. This change was negligible in comparison to the unlabeled oligonucleotide hybridization curve. These samples were allowed to hybridize at room temperature before being introduced into the channel and recording the signal for 60 seconds. The averaged signal from each sample was then normalized by subtracting the buffer signal, and the normalized signal was then plot versus target concentration (FIG. 15). These curves were then fit to a one-site binding hyperbola to obtain K_{D}.

TABLE 1
K_{D} values and standard error of free-solution BSI and surface-
immobilized BSI with corresponding melting temperature (Tₘ)

| | Free- | | | |
|---|---|---|---|---|
| | Solution BSI | Surface-Immobilized | Tₘ^{[b]} | Tₘ^{[c]} |
| DNA Strand | K_{D}-(nM)^{[a]} | BSI K_{D} (nM)^{[a]} | (° C.) | (° C.) |
| Unlabeled | 27.5 ± 4.7 | 66.1 ± 8.4 | 74.4 ± 0.3 | 72.2 |
| Cy3-labeled | 19.6 ± 5.8 | 41.7 ± 6.3 | 74.1 ± 0.1 | — |
| FITC-labeled | 17.4 ± 2.9 | 45.6 ± 7.7 | 75.2 ± 0.5 | — |
| Terminal | 28.9 ± 2.4 | 60.7 ± 7.9 | 73.1 ± 0.3 | 71.8 |
| Mismatch | | | | |
| Internal | 110.4 ± 11.6 | 132.5 ± 16.6 | 67.5 ± 0.2 | 66.7 |
| Mismatch | | | | |

^{[a]}Curve fits were carried out on an average plot from at least 5 separate trials to ensure reproducibility
^{[b]}Experimental Tm from absorbance hyperchromicity
^{[c]}Calculated Tm from DINAMelt two-state hybridization model

The free-solution experiments shown graphically in FIG. 15 and numerically in Table 1 provided interesting results. The presence of either fluorescent tag provided a modest stabilization of the duplex as shown by a lower K_{D}. These results were in agreement with previously published reports that show both the 5′-Cy3 and FITC labels lead to duplex stabilization. Furthermore, Norman et. al. have shown that when the Cy3 fluorophore in a 5′-labeled duplex is positioned on the end of the helix it mimics the addition of another base pair, thus increasing duplex stability. In comparison to the perfect complement, the terminal CA/AC mismatch had a negligible effect on duplex stability whereas the internal AG/GT mismatch had a deleterious impact on DNA hybridization. These results were consistent with previous reports that show stable terminal mismatches have a negligible impact on hybridization whereas internal mismatches significantly perturb hybridization.

Melting studies were carried out to obtain the Tm to compare the BSI free-solution data with a widely used method for determining duplex stability (Table 1). Note that direct comparison between Tm and K_{D} (ΔG) can be inaccurate when assuming a two-state model for oligomers over 14 bases long. As a consequence, the K_{D} of a duplex at room temperature may not correlate with Tm. Although the Cy3 and FITC labels lowered the K_{D} of hybridization as determined by BSI, their impact on duplex melting was small and within experimental error as shown by the relatively small change in Tm. Studies on mismatches showed lower melting temperatures than the perfect complement with a modest decrease in Tm for the terminal mismatch and a significantly lower Tm for the internal mismatch. Nearest neighbor calculations used to estimate the Tm for the complementary strand and mismatches correlated well with the experimentally determined Tm values. Comparison of data obtained from BSI were consistent, with Tm values of the terminal mismatch showing a slightly lower Tm and slightly higher K_{D} in comparison to the perfect complement, and the internal mismatch with a Tm five degrees lower than the perfect complement. Within experimental error of these experiments, there was reasonable correlation between Tm data and K_{D} values (r2=0.97).

The impact of surface immobilization on hybridization was examined. Surface-immobilized BSI experiments were carried out by immobilizing ExtrAvidin to the glass surface and reacting with the biotinylated Ps oligonucleotide. The lowest concentration of the target strand was then introduced into the channel and the signal recorded for 1.5 minutes. After the first 40 seconds the signal leveled out, signifying the hybridization was complete. However, only the last 1000 data points (˜the last 30 seconds) were used in the average to ensure the system was at equilibrium. After each hybridization event the channel was washed with 2×1 μL 1M NaOH followed by 3×1 μL MQ-H2O to rinse away the previous strand. The next highest concentration was then added to the channel and the procedure repeated until all concentrations had been recorded. The immobilization and wash procedure was validated by fluorescence microscopy with the use of the Cy3 and FITC-labeled oligonucleotide to ensure proper Ps immobilization and target strand removal. The binding curves were normalized by subtracting the buffer signal from the individual target signals and plot versus concentration (FIG. 16). Data analysis was carried out in the same manner as the free-solution experiments, using a one-site binding hyperbola, since the signal was recorded at equilibrium. These plots show a clear saturation by 200 nM with the exception of the internal mismatch which required a slightly higher concentration to reach saturation. The internal mismatch also provided a marked signal increase in comparison to the other strands. It was possible that the presence of an internal mismatch skewed the conformation of the duplex generating a greater signal shift. Smith and coworkers found that one-base bulges neighboring a mismatch can be present in several conformations, which can be easily interconverted.

Results from these experiments show a significant perturbation in the hybridization affinity for surface-bound versus free-solution oligonucleotides (Table 1). These perturbations result in a drastic increase in K_{D} values by nearly 50% when compared to free-solution values. It is important to note that while the surface-immobilized K_{D} values were substantially higher than free-solution, the relative order of stability of each pair remains the same. This suggested that although the absolute K_{D} values were perturbed in the surface-immobilized experiments, the trend in relative affinities remained the same. Comparison of free-solution and surface-immobilized BSI data showed that the surface-bound experiments generated a greater signal shift. This was most likely due to differences in Ps concentration in the surface immobilization format.

Overall, these studies showed that BSI provided an approach for the study of DNA hybridization in free-solution and surface-immobilized formats. Using this approach, the influence of terminal fluorophores and a two-base-pair terminal mismatch were shown to have a negligible or modest impact on hybridization of a 30-mer whereas a two base pair internal mismatch is significantly destabilizing. A direct comparison of surface-immobilized and free-solution hybridization was carried out using the same technique. Comparison of both formats showed that surface immobilization perturbed hybridization altering K_{D} values by as much as 50%. The simplicity of the experimental design and ease of use makes this approach broadly applicable for the study of a range of biomolecular interactions.

Oligonucleotide Preparation

All oligonucleotides were purchased salt-free with PAGE purification from BioSynthesis, Inc. The static strand (first nucleic acid polymer) was derived from the myosin binding region of the mouse actin gene (5′-ACTCATCGTACTCCTGCTTGCTGATCCACA-3′) (SEQ ID NO. 7). This strand was also tagged with a 5′ biotin for the surface studies. The mismatch strands (second nucleic acid polymer) contained a two base terminal mismatch (5′-CATGGATCAGCAAGCAGGAGTACGATGAGT-3′) (SEQ ID NO. 8) or a two base internal mismatch (5′-TGTGGATCAGCAAGAGGGAGTACGATGAGT-3′) (SEQ ID NO. 9). The complementary strand (5′-TGTGGATCAGCAAGCAGGAGTACGATGAGT-3′) (SEQ ID NO. 10) was then left unlabeled or 5′ labeled with either fluorescein isothiocyanate (FITC) or cyanine-3 (Cy3). The lyophilized DNA was reconstituted in PBS 0.05% Tween 20 buffer to a stock solution of 100 μM and the concentration double checked with UV absorbance at 260 nm.

### BSI Channel Preparation

In order to prevent nonspecific DNA interactions with the glass surface for free-solution studies, the channel was silanized. Prior to silanization, the channel was cleaned by soaking in concentrated H2SO4 for 60 minutes followed by a MQ-H2O rinse and dried with compressed air. The channel was then filled with 2% 3-mercaptopropyltriethoxysilane (MEPTES, ordered from Sigma Aldrich) in toluene and allowed to sit without drying out for 60 minutes. The channel was then rinsed with toluene, methanol, and MQ-H2O to complete the silanization procedure.

The channel was prepared for surface-immobilization experiments by cleaning thoroughly with a 60 minute H2SO4 soak followed by a 30 minute 10% KOH in methanol soak. After rinsing with MQ-H2O and drying with compressed air, the channel was silanized with a 60 minute 2% MEPTES in toluene soak followed by a toluene, methanol, and H2O rinse and air dried. This step was followed by 1 mM N-(γ-maleimidobutyryloxy)succinimide ester (GMBS, ordered from Sigma Aldrich) in absolute ethanol for 30 min. Once again, the channel was rinsed with deionized water and dried before soaking in an ExtrAvidin (from Sigma Aldrich) solution (1 mg/mL) overnight. Having coated the channel with ExtrAvidin the biotinylated oligonucleotide (1 mg/mL) was immobilized onto the surface by soaking for 60 minutes.

BSI Experimental Protocol

The HeNe laser and temperature controller were turned on at least an hour before the experiments were conducted to ensure equilibrium. Free-solution experiments were carried out by keeping one strand constant at 150 nM and varying its complementary or mismatched strand from 0-300 nM. The DNA samples were allowed to mix for 4 hours at room temperature prior to the experiment to ensure complete hybridization. Samples were introduced by pipetting 1 μL directly into the channel well and using vacuum suction. Prior to each trial, buffer was rinsed through the channel until the signal remained constant. Each trial consisted of a one minute recording of the signal at each concentration. The signal values from these trials were then averaged and plot versus concentration. The corresponding plot was then fit to a one-site binding hyperbola model in Prism® to obtain K_{D} and standard error was determined in the curve fit analysis.

For the surface-immobilized BSI experiments, the single stranded oligomer was introduced into the prepared channel and the signal recorded for 1.5 minutes. Only the last 1000 data points were used in the average to ensure a uniform signal and avoid the signal change due to the hybridization event. After each hybridization event the channel was washed with 2×1 μL 1M NaOH followed by 3×1 μL MQ-H2O to rinse away the previous strand. The next highest concentration was then added to the channel and the procedure repeated until all concentrations have been recorded. Data analysis was the same as for the free-solution since the signal average was recorded after hybridization occurred.

Measurement of Melting Temperature

Oligonucleotide melting temperature (Tm) experiments were carried out on the JASCO CD Spectrophotometer. Samples were prepared by mixing equivolumes of 100 μM DNA solutions and allowing to hybridize for an hour. This solution was then diluted to a final duplex DNA concentration of 5 μM. The temperature was increased from 40° C. to 90° C. in 0.5° C./minute increments and monitored at 260 nm to obtain a melting profile. Tm was calculated in Origin® (Microcal Version 5) as the derivative of the melting curves and standard error was calculated from the average of 3 separate trials.

Data Analysis

End-point determination of K_{D} is straightforward and fairly simple since all measurements are taken after the binding complex is already formed. According to the law of mass action where [R]₀ is the initial macromolecule concentration, [L]₀ is the initial ligand concentration, and [R·L]_{∞} equals the receptor-ligand concentration at equilibrium, the following is true:

$${[ R \cdot L ]}_{∞} = \frac{{[ R ]}_{0} \cdot {[ L ]}_{0}}{{[ L ]}_{0} + {K}_{D}}$$

This equation can also be fit in Prism® using the one site binding hyperbola function (eqn 2) to directly find K_{D} from a plot of signal vs. ligand concentration. Y=(Bₘₐₓ·x)/(K_{D}+X)

### Example 4

Experiments similar to Example 3 were conducted. BSI measured the change in refractive index of a solution as two molecules bound. A back-scattered interference fringe pattern was generated when the beam of a laser is directed through a microfluidic chip containing the sample solution and then reflected back to a CCD detector. The hybridization of small strands of DNA was studied with BSI, and binding curve data were analyzed using Microsoft Excel and GraphPad Prism 4. In Example 3, 30-base pair oligonucleotides were used; in Example 4, 20-base pair oligonucleotides were used. For these DNA hybridization trials, a probe strand was held at a constant 100 nM concentration in PBS buffer while binding strands have a concentration range of 25 nM to 250 nM. Binding curves and K_{D} values for the strands' interactions are obtained. The probe strand is a 20-base pair sequence of actin, and the first binding strand studied was a perfect compliment.

TABLE 1
KD values of DNA binding curves

| Strand | K_{D} Values | K_{D} Error | R² |
|---|---|---|---|
| Comp | 22.42 | 2.697 | 0.9936 |
| Mis M | 43.31 | 3.806 | 0.9964 |
| Del M | 33.49 | 2.259 | 0.9977 |
| Del Q | 15.93 | 3.165 | 0.986 |

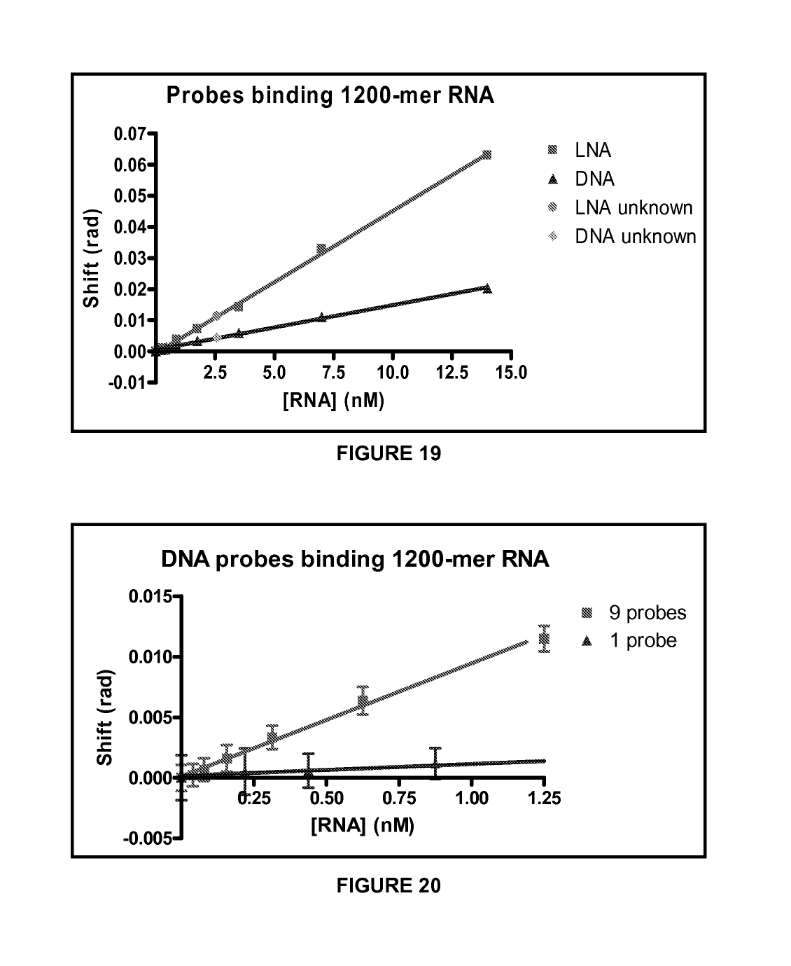

Once the complimentary binding curve was obtained, other strands featuring a single nucleotide polymorphism (SNPs; either a mismatched or deleted base pair) were studied. New binding curves for each SNP can be compared to that of the complimentary strand using the K_{D} values in order to gauge how the SNP affects the hybridization. A lower K_{D} corresponds to tighter binding, thus, a strand that was not a perfect complement to the actin would be expected to have a higher K_{D} value than the complementary strand, since the SNP would disrupt and weaken the strands' interactions. So far this has been seen in two of the SNP strands: one with an A/G mismatch in the middle of the strand, and one with a T deleted in the middle of the strand. In both cases, the K_{D} increased from 22.42 (complementary binding) to 43.31 and 33.49, respectively. FIG. 19 shows the binding curves of four of the strands, and Table 1 summarizes the results. FIG. 19 shows the binding curves of DNA hybridization obtained using BSI.

The current strand studied was one in which a T was deleted closer to the end of the strand, five base pairs in (indicated as ‘quarter deletion’). A deletion or mismatch in this area would be expected to still have an effect on the binding, but less the extent of the SNPs located in the middle of the strands. A rough binding curve and K_{D} value for this strand's binding to actin has been obtained, but more trials are still needed to decrease the error in some of the points.

The BSI technique demonstrated that it is sensitive enough to detect even a single mismatched nucleotide in a strand of DNA, and also allowed for the comparison of various SNPs in strands, which could prove useful for diagnostics and screenings. In addition, the BSI required a sample size many times smaller than traditional methods such as isothermal titration calorimetry (ITC), a method also used to study the binding of two molecules based on thermodynamic parameters (a microliter compared to a milliliter), reducing waste and material expenses.

The BSI technique demonstrated that it is sensitive enough to detect even a single mismatched nucleotide in a strand of DNA, and also allowed for the comparison of various SNPs in strands, which could prove useful for diagnostics and screenings. In addition, the BSI required a sample size many times smaller than traditional methods such as isothermal titration calorimetry (ITC) (a microliter compared to a milliliter), reducing waste and material expenses.

(SEQ ID NO. 11)
Actin Strand: 5′ [symbol] -ACTCATCGTACTCCTGCTTG-3′
(SEQ ID NO. 12)
Complementary: 5′-CAAGCAGGAGTACGATGAGT-3′
(SEQ ID NO. 13)
Middle A/G Mismatch: 5′-CAAGCAGGAGGACGATGAGT-3′
(SEQ ID NO. 14)
Middle Deletion: 5′-CAAGCAGGAGACGATGAGT-3′
(SEQ ID NO. 15)
Quarter A/G Mismatch: 5′-CAAGCAGGAGTACGAGGAGT-3′
(SEQ ID NO. 16)
Quarter Deletion: 5′-CAAGCAGGAGTACGAGAGT-3′

### Example 5

### Detecting Short Strands of RNA and Full-Length RSV N-Gene

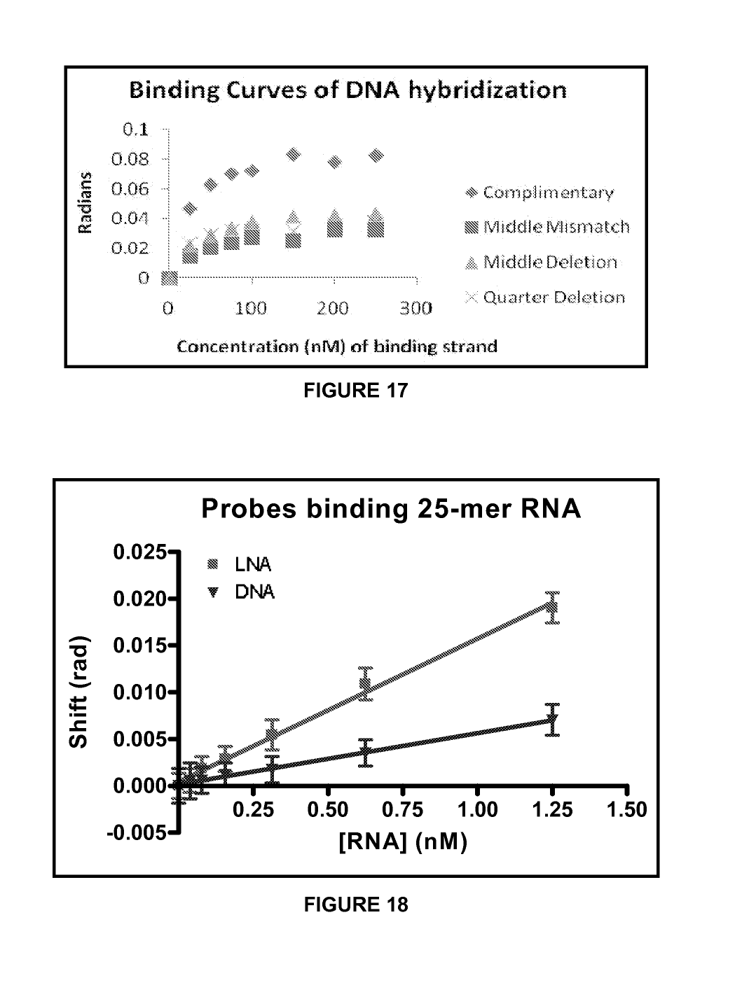

Hybridization assays, using a BSI device as disclosed herein, was used to detect short strands of RNA. The hybridization assays were prepared by incubating a constant, excess concentration of either DNA or LNA probe with various concentrations of target RNA overnight before detection was carried out. See FIG. 17 for the results of the hybridization assays. FIG. 17 shows standard curves of a synthetic 25-mer RNA target quantified by binding with a complementary probe of DNA (squares) and a locked nucleic acid (LNA) probe (triangles) that used a modified nucleotide having an extra bridge connecting the 2′ oxygen and the 4′ carbon. The bridge locks the ribose in the 3′ endo (North) conformation. The DNA and LNA probes were used to detect a 25 base pair section of RNA from RSV (respiratory syncytial virus) n-gene. The DNA level of detection was 1.75±0.29 nM and the LNA level of detection was 0.419±0.003 nM. The LNA probe showed increased signal and four-fold improvement in level of detection.

The full-length RSV N-gene was also detected using BSI methods. The results for the measured concentrations of an unknown sample of RSV target obtained using BSI were in close agreement with those obtained with PCR. See FIG. 19. The “unknown” actual concentration (by PCR)=2.59 nM. “Unknown” observed concentration (single DNA or LNA probe)=2.59±1.22 nM. “Unknown” observed concentration (single LNA probe)=2.71±0.51 nM. DNA LOD (level of detection)=1.37±0.24 nM. LNA LOD=0.862±0.130 nM. Standard curves of a 1200-mer RNA target quantified by binding with a 25-mer DNA or LNA. An “unknown” was prepared by spiking an aliquot of cell lysate with RSV RNA. The sample was treated to purify the RNA. Results of the hybridization of the sample with the 25mer DNA and LNA are shown in FIG. 19.

TABLE II
Results

| | Short 25 b.p. | Full Length 1200 b.p. |
|---|---|---|
| | RNA target | RNA target |
| DNA probe | LOD = 1.75 nM | LOD = 1.37 nM |
| | Slope = 0.0055 | Slope = 0.0014 |
| LNA probe | LOD = 0.42 nM | LOD = 0.86 nM |
| | Slope = 0.0153 | Slope = 0.0046 |

From the data, it appeared that BSI LOD=1×10⁶ RNA molecules in the probe volume. LOD for standard PCR methods is 1×10⁸ molecules in a 1 mL sample. Required detection limit for 1 mL patient sample at the IC₅₀ level of detection is 1×10⁵ molecules. With the BSI methods and devices of the present invention, and optionally pre- and/or post-treatment of samples and/or probes, BSI LOD is 1.3×10³ to 6.7×10³. BSI methods and devices can quantitatively detect low concentrations of RNA using DNA, LNA or RNAA probes. LNA probes provided improved S/N (signal to noise) ratio and LOD over DNA probe. BSI methods and devices can detect RNA targets ranging from 25 to at least 1200 base pairs while maintaining low nanomolar detection limits. In combination is certain aspects of the invention, such as multiple and surface immobilization, a thirty-fold improvement in LOD was seen. LOD of 1×10⁶ is two orders of magnitude power than concentrations found in typical patient samples.

### Example 6

### Multiple Probes

The full-length RSV n-gene is 1200 base pairs and there are numerous unique, non-overlapping regions that can be the target of multiple probes. In this example, nine unique 25-mer DNA probes were used to detect the n-gene. See FIG. 20. Immobilization of the probes to the channel surface allowed the sample containing the n-gene to be cycled over the surface more than one time. A majority of the target molecules in the sample were captured. The nine probes LOD=0.62±0.06 nM. The 1 probe LOD=1.37±0.024 nM. Using a mixture of nine probes, having differing sequences, resulted in a six-fold increase in signal and a two-fold improvement in LOD over using a single DNA probe. See FIG. 21 for a schematic of the capture of molecules in a BSI channel.

Table of Probes Used:

| Position | | SEQ |
|---|---|---|
| in N | | ID |
| gene mRNA | Probe sequence (5′-3′) | NO. |
| 4-29 | GCTCTTAGCAAAGTCAAGTTGAATGA | SEQ ID |
| | | NO. 17 |
| 193-214 | ATAGGTATGTTATATGCGATGT | SEQ ID |
| | | NO. 18 |
| 242-263 | AAATACTCAGAGATGCGGGATA | SEQ ID |
| | | NO. 19 |
| 425-444 | TGGGAGAGGTAGCTCCAGAA | SEQ ID |
| | | NO. 20 |
| 531-551 | TGGTCTTACAGCCGTGATTAG | SEQ ID |
| | | NO. 21 |
| 603-622 | ACCCAAGGACATAGCCAACA | SEQ ID |
| | | NO. 22 |
| 755-774 | GTGCAGGGCAAGTGATGTTA | SEQ ID |
| | | NO. 23 |
| 843-872 | GGAACAAGTTGTTGAGGTTTATGAATATGC | SEQ ID |
| | | NO. 24 |
| 957-983 | CTTCTCCAGTGTAGTATTAGGCAATGC | SEQ ID |
| | | NO. 25 |

### Example 7

### Binding of Mannose by Concanavalin A

See FIG. 22. This experiment was designed to compare the BSI signal generated by mannose binding concanavalin A (ConA), where ConA is either in free-solution or immobilized using avidin-biotin coupling. Even without cycling the sample over the immobilized biological molecule, surface immobilization made for a three-fold increase in binding signal in a BSI device. Conditions and materials for the experiments were substantially the same as those taught in Baksh, M., et al., Nature Biotechnolgoy 29, 357-360 (2011) and Kussrow, et al., Anal. Chem., 2009, 81, 4889-4897, each of which is incorporated herein in its entirety.

### Example 8

### Multiplex Design BSI Methods and Devices

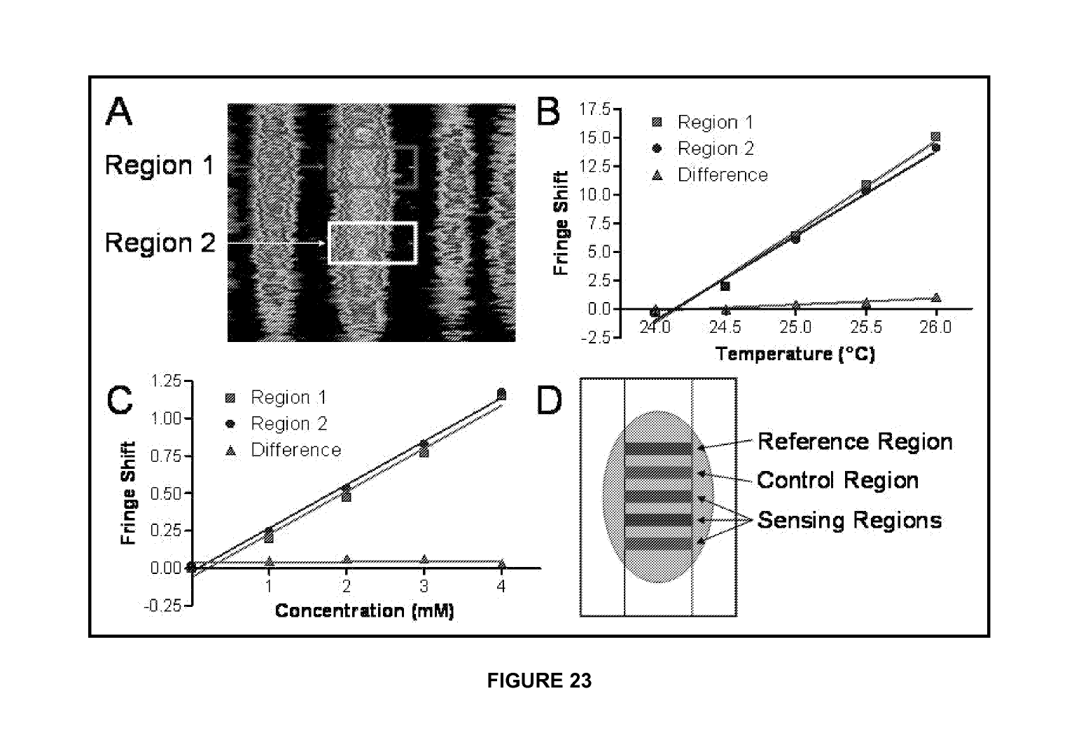

BSI methods and devices comprise multiplex designs. For example, a BSI device may have several detection regions, including but not limited to, a reference region, which provides the baseline measurement for binding of two or more biological molecule species; a control region, where positive and negative controls are provided; and sensing regions, where unknown biological molecule species bind to known probes or binding partners. FIG. 23 A shows the fringes resulting from water at Region 1 and 2, which are two regions in the channel of a BSI device. FIG. 23 B shows a graph resulting from solutions of glycerol in Regions 1 and 2 being subjected to changes in temperature. FIG. 23 C shows a graph resulting from the solutions in Regions 1 and 2 changing to sequentially increasing concentrations of glycerol. FIG. 23 D shows a schematic of a multiplex design of a channel of a BSI device.

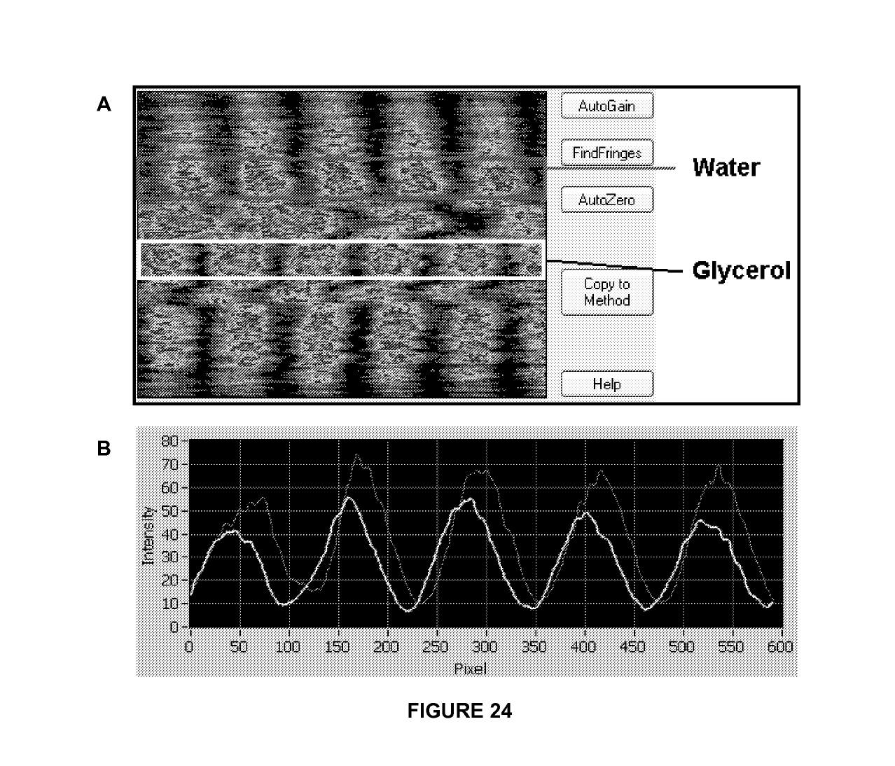

FIG. 24 A shows a screen shot of a computer screen of fringes of glycerol and water. This figure shows that two distinct sample regions are easily detected, and do not interfere with each other. FIG. 24 B shows the corresponding line profile of the fringes to be used in the FFT analysis. Because the camera is a 2D CCD array, the signal from multiple rows can be averaged, reducing the short term signal variation due to noise.

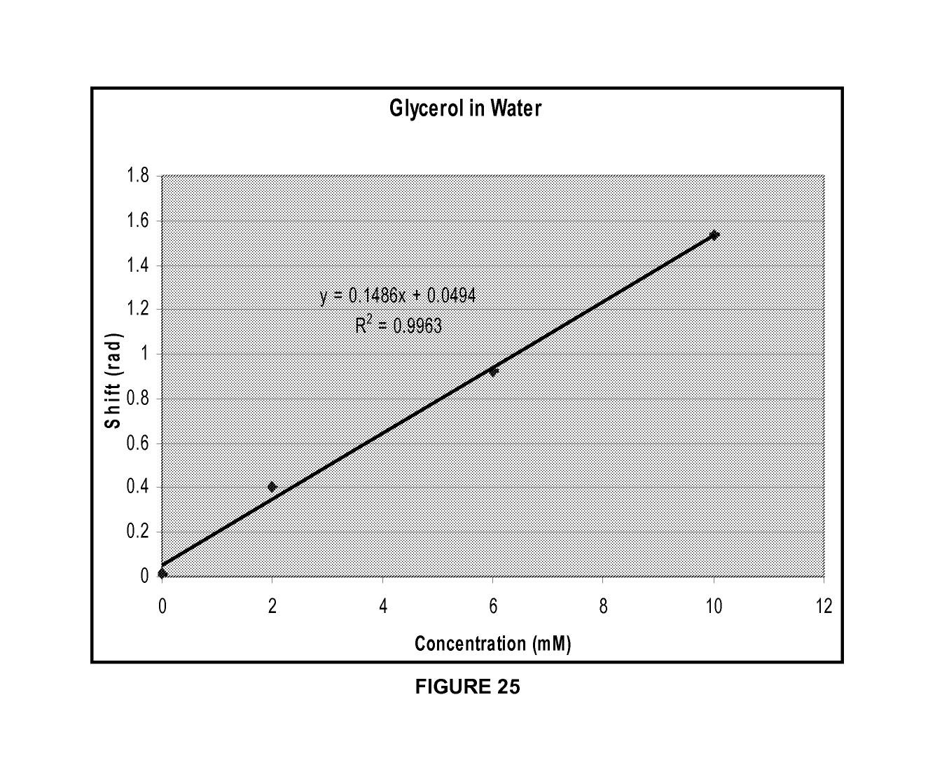

FIG. 25 shows data from a multiplex BSI wherein surface tension values were established. Using the multiplex BSI device without a temperature controller, the LOD for glycerol in water is 0.042 mM (error bars are present). Normally, a temperature controller is required to prevent a drift in the BSI signal due to temperature variations. Because the multiplex design has an internal reference, the affect of temperature changes on the signal are minimized. Typical limits of detection for the non-multiplex device were 0.2 to 0.5 mM. See FIG. 25.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A method comprising detecting nucleic acid polymer sequence mismatch with a target nucleic acid polymer by hybridization of the target nucleic acid polymer with at least one nucleic acid probe comprising a known sequence, and back-scattering interferometry.

2. The method of claim 1, wherein the target nucleic acid polymer is made by PCR.

3. The method of claim 1, wherein the target nucleic acid is present in a concentration of less than about 5.0×10⁻⁷M.

4. The method of claim 1, wherein the target nucleic acid is present in a concentration of less than about 5.0×10⁻⁹M.

5. The method of claim 1, wherein at least one nucleic acid probe is not bound to a substrate.

6. The method of claim 1, wherein at least one nucleic acid probe is bound to a substrate.

7. The method of claim 1, wherein the nucleic acid polymer hybridization occurs in a sample in a microfluidic channel in a substrate and back-scattering interferometry comprises directing a coherent light beam onto the substrate such that the light beam is incident on the channel to generate backscattered light through reflective and refractive interaction of the light beam with a substrate channel interface and the sample, the backscattered light comprising interference fringe patterns including a plurality of spaced light bands whose positions shift in response to changes in the refractive index of the fluid sample.

8. The method of claim 1, wherein a phase wrap occurs when the fringes move more than one cycle (π), further comprising correcting for the phase wraps using the equation:
Adjusted Value=−π−(π−signal)−n(2π).

9. A method for detecting a target nucleic acid in a sample comprising,
a) providing a substrate having a channel formed therein for reception of a fluid sample to be analyzed;
b) introducing a target nucleic acid in a sample into the channel;
c) providing at least one nucleic acid polymer probe with a known sequence;
d) providing hybridization conditions in the channel;
e) directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; and
f) detecting positional shifts in the light bands.

10. The method of claim 9, the target nucleic acid is from a polymerase chain reaction (PCR) after 1, 2, 3, 4, 5, 6, 7, 8, 9, or 10 cycles.

11. The method of claim 9, wherein at least one nucleic acid probe is not bound to a substrate.

12. The method of claim 9, wherein at least one nucleic acid probe is bound to a substrate.

13. The method of claim 9, wherein a phase wrap occurs when the fringes move more than one cycle (a), further comprising correcting for the phase wraps using the equation:
Adjusted Value=−π−(π−signal)−n(2π).

14. The method of claim 1, wherein the substrate and channel together comprise a capillary tube.

15. The method of claim 1, wherein the substrate and channel together comprise a microfluidic device.

16. The method of claim 15, wherein the microfluidic device comprises a polymeric substrate and an etched channel formed in the substrate for reception of a fluid sample, the channel having a cross sectional shape.

17. The method of claim 16, wherein the polymeric substrate comprises one or more polymers selected from polycarbonate, polydimethylsiloxane, fluorosilicone, polytetrafluoroethylene, poly(methyl methacrylate), polyhexamethyldisilazane, polypropylene, starch-based polymers, epoxy, and acrylics.

18. A method for detecting a target nucleic acid in a sample comprising,
a) providing a substrate having a channel formed therein for reception of a fluid sample to be analyzed;
b) introducing a sample comprising a target nucleic acid polymer;
c) providing one or more oligonucleotide probes with sequences that have from 1 to ten nucleotide bases different from the target nucleic acid and that hybridize to the target nucleic acid polymer;
d) directing a coherent light beam onto the substrate such that the light beam is incident on the channel to generate backscattered light through reflective and refractive interaction of the light beam with a substrate/channel interface and the sample, the backscattered light comprising interference fringe patterns including a plurality of spaced light bands whose positions shift in response to changes in the refractive index of the fluid sample; and
e) detecting positional shifts in the light bands;
wherein the positional shifts in the light bands detect the extent of hybridization of the one or more probes to the target nucleic acid polymer.

19. The method of claim 18, wherein detecting of positional shifts occurs in more than one location.

20. The method of claim 18, wherein at least one nucleic acid probe is not bound to a substrate.

21. The method of claim 18, wherein at least one nucleic acid probe is bound to a substrate.

22. An apparatus comprising a backscattering interferometer and a thermocycler.

23. A method for detecting the location of a sequence mutation in a target nucleic acid sequence, comprising providing a BSI device having at least a first nucleic acid polymer probe immobilized in the channel, wherein the probe sequence is known; introducing a composition comprising a non-immobilized nucleic acid polymer composition comprising target sequence nucleic acid polymers into the channel, wherein the immobilized nucleic acid polymers interact under hybridization conditions with the non-immobilized nucleic acid polymers; directing a coherent light beam to generate backscattered light comprising interference fringe patterns including a plurality of spaced light bands which positions shift in response to changes in the refractive index of the compositions and hybridization products in the channel; detecting positional shifts in the light bands relative to a baseline; and determining the formation of one or more hybridization products of the immobilized nucleic acid polymer with the non-immobilized nucleic acid polymer from the positional shifts of the light bands in the interference patterns.

24. The method of claim 23, wherein detecting of positional shifts occurs in more than one location.

25. The method of claim 23, wherein the positional shifts of the light bands in the interference patterns are used to determine the K_{D} of the probe and target sequence.

26. The method of claim 23 further comprising repeating steps a)-d) one or more times, with one or more probes, each with a known sequence that is a derivative sequence of a known probe, wherein the derivative probe(s) have mismatch bases in differing locations.

27. The method of claim 23, wherein the positional shifts of the light bands in the interference patterns are used to determine the K_{D} of each of the probes and the target sequence.

28. The method of claim 27, wherein a K_{D} close in number to the K_{D} of a hybridization product having 100 percent homologous sequence indicates substantially homologous hybridization between the probe and the target sequence.

29. The method of claim 27. wherein a K_{D} higher in number to the K_{D} of a hybridization product having 100 percent homologous sequence indicates mismatch base pairing hybridization between the probe and the target sequence.

30. The method of claim 9, wherein the substrate and channel together comprise a capillary tube.

31. The method of claim 9, wherein the substrate and channel together comprise a microfluidic device.

32. The method of claim 31, wherein the microfluidic device comprises a polymeric substrate and an etched channel formed in the substrate for reception of a fluid sample, the channel having a cross sectional shape.

33. The method of claim 32, wherein the polymeric substrate comprises one or more polymers selected from polycarbonate, polydimethylsiloxane, fluorosilicone, polytetrafluoroethylene, poly(methyl methacrylate), polyhexamethyldisilazane, polypropylene, starch-based polymers, epoxy, and acrylics.


SEQUENCE LISTING (25 sequences)

SEQ ID NO: 1 - DNA, 13 bp, Artificial Sequence
  Description of Artificial Sequence note = synthetic construct
atcgggttaacct

SEQ ID NO: 2 - DNA, 26 bp, Artificial Sequence
  Description of Artificial Sequence note = synthetic construct
aggttaacccgataggttaacccgat

SEQ ID NO: 3 - DNA, 13 bp, Artificial Sequence
  Description of Artificial Sequence note = synthetic construct
aggttaacttgat

SEQ ID NO: 4 - DNA, 12 bp, Artificial Sequence
  Description of Artificial Sequence note = synthetic construct
aggttaaccgtt

SEQ ID NO: 5 - DNA, 13 bp, Artificial Sequence
  Description of Artificial Sequence note = synthetic construct
aggttaacttgat

SEQ ID NO: 6 - DNA, 13 bp, Artificial Sequence
  Description of Artificial Sequence note = synthetic construct
aggttaacttgat

SEQ ID NO: 7 - DNA, 30 bp, Artificial Sequence
  Description of Artificial Sequence note = synthetic construct
actcatcgtactcctgcttgctgatccaca

SEQ ID NO: 8 - DNA, 30 bp, Artificial Sequence
  Description of Artificial Sequence note = synthetic construct
catggatcagcaagcaggagtacgatgagt

SEQ ID NO: 9 - DNA, 30 bp, Artificial Sequence
  Description of Artificial Sequence note = synthetic construct
tgtggatcagcaagagggagtacgatgagt

SEQ ID NO: 10 - DNA, 30 bp, Artificial Sequence
  Description of Artificial Sequence note = synthetic construct
tgtggatcagcaagcaggagtacgatgagt

SEQ ID NO: 11 - DNA, 20 bp, Artificial Sequence
  Description of Artificial Sequence note = synthetic construct
actcatcgtactcctgcttg

SEQ ID NO: 12 - DNA, 20 bp, Artificial Sequence
  Description of Artificial Sequence note = synthetic construct
caagcaggagtacgatgagt

SEQ ID NO: 13 - DNA, 20 bp, Artificial Sequence
  Description of Artificial Sequence note = synthetic construct
caagcaggaggacgatgagt

SEQ ID NO: 14 - DNA, 19 bp, Artificial Sequence
  Description of Artificial Sequence note = synthetic construct
caagcaggagacgatgagt

SEQ ID NO: 15 - DNA, 20 bp, Artificial Sequence
  Description of Artificial Sequence note = synthetic construct
caagcaggagtacgaggagt

SEQ ID NO: 16 - DNA, 19 bp, Artificial Sequence
  Description of Artificial Sequence note = synthetic construct
caagcaggagtacgagagt

SEQ ID NO: 17 - DNA, 26 bp, respiratory syncytial virus
gctcttagcaaagtcaagttgaatga

SEQ ID NO: 18 - DNA, 22 bp, respiratory syncytial virus
ataggtatgttatatgcgatgt

SEQ ID NO: 19 - DNA, 22 bp, respiratory syncytial virus
aaatactcagagatgcgggata

SEQ ID NO: 20 - DNA, 20 bp, respiratory syncytial virus
tgggagaggtagctccagaa

SEQ ID NO: 21 - DNA, 21 bp, respiratory syncytial virus
tggtcttacagccgtgattag

SEQ ID NO: 22 - DNA, 20 bp, respiratory syncytial virus
acccaaggacatagccaaca

SEQ ID NO: 23 - DNA, 20 bp, respiratory syncytial virus
gtgcagggcaagtgatgtta

SEQ ID NO: 24 - DNA, 30 bp, respiratory syncytial virus
ggaacaagttgttgaggtttatgaatatgc

SEQ ID NO: 25 - DNA, 27 bp, respiratory syncytial virus
cttctccagtgtagtattaggcaatgc